SEQUENCE LISTING (1 sequence)

SEQ ID NO: 1 - DNA, 61065 bp, Homo sapiens
taaatgatgatgttggaatagattggaccaatgaaaatgatcgtctactggtttgtcaaatgtgtataaagttggctgatatcaatggtccagctaaatgtaaagaactccatcttcagtggacagatggtattgtcaatgaattttatgaacaggtaactgaccactgtttaatacagcttaatctgtacttacaggttgctcatgaattgctcaaagcttctaacagctatgaaagtatgatcactctattttatattcatacttttgtcagaaaatgcactggtcaattttgatcaggagacaaaaagtgacgtataaaaattggactaaaatataatcatagaactctatgataaaagttatttgcaagcatttgggggaagaagatattgagatagtggaagtcagatgttaggaagatccttgaaatactgtttttttatagatttatttattatcatagaaggtagtttaaatgtatatggagtagcttgtatgtatatggtagtttaaatgtatatgcatatcacattaatttcacttactttcctaaaaaatttatttattatcatagaaggtagtttaaatgtacatggaatagtttatatgtatatgtatatggtagttcgaatgtatctgtatattagattaacttcacttttgtaaaaaaattaataagagcattatgtgaaaaagctaaaaagaaatagacatttcatttgacatgataactaagagttaacagactactcagaaagctaccacaattacataagctgggggtgatcctcctgacattaccctacacccatcagaatgcacttgaaccttcctgtttggcactgtgaaccactcttaaaaagaatgtggaggtgaaggagcatgcataaaggacaggaataagtgacagaaaagcatgtggaaaaaaatgaaggtatttactcttggggaaatataactgtcttcagatatttaaaagagtgacatactaatttagaaatcggttaatcaacattgctgtggagattaaagaagataaattatataacatacttagcacaggatctgattcataaaaaaagttagatccctctctactgccctaaaaaaggtatgaatataccagtctatagaagttattgggaggtagtgttcaggtcaatgtcagaaagaaatttatattgctaaaggtatccaaaaaataggctgagacaccatattaatggtaataaattgactgtcattggacatgtttaaacaagagattagaatgaaattttatggttactataggtaggattcatatatagaatgaaagattttttttattttattatactttaagttttagggtacatgtgcacaacgtgcaggtttgttacatatatatacatgtgccctgttggtgtgctgcgcccattaactcatcatttagcattagatatatctcctaatgctatccctccctcctccccccaccccacaacagtccccagtgtgtgatgttccccttcctctgtccatgtgttctcattgttcagttcccacctatgagtgagaacatgcggtgtttggttttttgtccttgcgatagtttgctgagaatgatggtttccagtttcatccatgtccctacaaaggacatgaactcatccttttttatggctgcatagtattccatggtgtatatgtgccacattttcttaatccagtctatcattgttggacatttgggttggttccaagtctttgctattgtgaatagtgcctcaataaacatacatgtgcatgtgtctttatagcagcatgatttgtagtcctttgggtatatacccagtaatgggatggctgggtcaaatggtatttctagttctagatccctgaggaatagccacactgacttccacagttgttgaactagtttacagtcccaccaacagtgtaaaagtgttcctatttctccacatcctctccagcacctgttgtttcctgactttttaatgattgccattctaactggtgtgagatggtatctcgttgtggttttgatttgcatttcactgatggccagtgatgatgagcattttttcatgtgtcttttggctgcataaatgtcttcttttgagaagtgtctgttcatatccttcacccactttttgatggggttgtttgtttttttcttgtaaatttgtttgagttcattgtagattctggatattagccctttgtcagatgagtaggttgcaaaaattttctcctaaaaccataaaaaccctagaagaaaacctaggcaataccattcaggacataggcatgggcaaggacttcatgtctaaaacaccaaaagcaatggcaacaaaaaccaaaattgacaaatggggatctcatcaaactaaagagcttctgcacagcaaaagaaactaccatcagagtgaacaggcaacctacagaatgggagaaagttttgtgtttttaaaacatcatgaaatcatgtattgcctttcctaagaatgggattaattcataaatctcgaaatatggaaatgcagaaaaatgtaaattaaagggaaaattcatgtataataataccacttcttaggcataattgttgctgatattttgggatatttcctttattaattttgctagataactgctggttaaaaatctcccttttttcctcagaaacatttgaagcacttcaaaataaaacatttgtacagtaatattattaaacatatagataaataagaatatcagaaaccacacaatcacaggagatatattaaaattcaaacaaaatctgggataaggtaaccccttgaattgagcaataaattctttaaagctttctggaagccaaagcaaaaaaaggaaaataggctacatttatatatttttaaaaaaattgttctcactattgcatttttttttttgagatggagtctcactctgtcaccctggaatgcagtggcacgatctcagctcaccgcaacctccgcctcctgggttcaagtgattctcctgcctcagcctcccaagtagctgggattacaggcacctgccactacacccagctaattttttgtatttttaatagagacggagtttcaccatgttggccaggctggtctcgatttgcccactttggcatcccaaagtgctgggattacaggcgtgagccaccacgcccagcctcaccattgcattttcgtgcatttatattctcatataaaagacactgacttgtgtgattaaaatatcatctacttatacattagaatttagactttctgtatttctccttgataaaaagcaaaaacctcaagttcagaaaagtactaattagcaacagtgtttctataggaaccttagcacttgaggtaggatcctttagctgtcaagtaaagaagtttgtgcattaatgcctggggttttacatatttacaaatttcagatgccaggtgaatgttgacttcacttgtattttatttctagggtgatgaagaggccagccttggattacccataagccccttcatggatcgttctgctcctcagctggccaaccttcaggaatccttcatctctcacattgtggggcctctgtgcaactcctatgattcagcaggactaatgcctggaaaatgggtggaagacagcgatgagtcaggagatactgatgacccagaagaagaggaggaagaagcaccagcaccaaatgaagaggaaacctgtgaaaataatgaatctccaagtaagttctaaaacctagttctaatgtgttttccctgggattgctttcttactttagatgaattttatgcttcttatgaaatacacaatacttcaagtctaaacatcatttgcggagtttgactacggatagctcccttccccaatatcataataatagaaatcccagactatggaatctataataataacacatatgatacaaagatatcttctccgtcatgaagtcatgaagttagaaaaaaataataataaggagttgaagcctattcttcagggtatctggttttttgtttgttttttgttttttgaggcagagtcttgctctgtcacccaggctggcgtgcagtggcacgatctcggctcactgcaagctccgcctcccaggttcatgccattctcctgcctcagcctcctgagtagctgggactacaggtgcgcaacactacacccggctttttttttttttttttttttttttgtattttgagtagagatcgggtttcaccatgttagccaggatggtctcgatctcctgaccttgtgatctgcctgcctcgacctcccaaagtgctgggattacaggcgtgagccaccgcgcccagctgcttcaggttatgttagcgatttaaaagtgaaagaatttcagttaccataaagaccagtcataggtttttattggcttggttaggggaactccatgtgaatagataggaggtagtattcatttgttcattcaacagacatttattgagcatctgctctgtgccaggtatccttccaggtactagagatagagttgtggggaaaaaaagacaaaaaataaaataaaatccttgcccttatagaagtacattctagtgggatcagtagacaataagcaaaacaagtgagtatatctatagtatgttaaatacagtattatttactgtagtgataaatgctgagaaatatgagagtaaaaataaaaggagggagatatgaaatattatagagataggttaaaattttaggtaagatatccagggataggaaagatgtaaagcagggaaagatttgtaaaaagaaaaagaaaagacctgcaagaagcaagggagtgctccctggggctgtatgtggaaagaggattccaaagagaggagaaagcaacctgtgcaaaggtccggggatgtacattgggtggggtgttcagggagcagcaggagaactgggatagctggagcagcaagacagaggcagccgggaacgaactgtccaagggagagaatacagtcatgggttcttagttgctgtttctggttgggccagtaaagccccttcctcatcccactgttctgcttatcactagagacagaaactaaaaaccatggtttcaggtgctaaaagcctaaaacaaaacaaaacagaagaacagcaacaacaaataagtcagcctggacaagcttggaaggtgggactctgtaggtcacggtaagtgttgtggcttttactctgagtaagatgggaaatcgtcagtggttttgaacggaagagttagtagactcaacttacattttaactggactgcttcagctgctctattgagaatatgcttggggttgagaagggagcaaggatagacatagggcggtcagttaggagaccattgaaattatccaagtgagagatgcctgtgactttgaaccagggcagtagcaacagtgacagtgacaacctatcacatgtgataattggctggagtgcatcatgtggccaatctaacctatttagcttaattgactatgaaatcatttttatttatgcctctaaagctactctcctatttatcctgtcatgaaacaaatagtgactaatagcataattagtaaaaatccatgacccttgcaatgatattgcacaaatactgctcctaagaacatagtatgtgatttctgtctacctttacagataaagtacaatcttaatatcctcaaggttcttaccaaaaatacaaccagtgaaagacattctgatcatttcgtgcctatttttatcacagtcaggataatgcacaacttggttcattccaaccatcttgtaaggagattaaccttctactgatccattttgctctcatataagcaccttgttgtaggcacacataagcaaacataaagaagtccttatcaattatgaacttatgaagtaaaattttgcaaattgagttttagatcagatctggcctgttaattactttttgtagaatgggggaaaattaaagttgtttttcttcaatatgctactgtcaaagtaattatatttcatttatctgatgcaaataaaaagacttccattagaaaatttgtactaatcttgagattcagagctagaagccaagacttggaattcatgggttattcattcatttttttcacttaataaacatgtattagttttctctttttgtttgttttcatttcaaagagccaggtagtttgctgtcttactaagaataagacagtttttccactgaatgaaggtattgtcagcttagcctctggaaaagctttgttcccaggggccctgacttactgctctttgaactcaaacacatggaaaatacagacgcacagatccaggcattgggaattgaaggacccaggccaggagacttaacaggctcaacttgtcaacctagcctttagcagcaaatgctacaaccacatattccatttctttctgttctgtagggttgggttgtcataccttccagtttttatcaagttcactaaagcaggaatgagagactttttgggttctgtttgctcatgccttccaaaagttaactgaccacattatattacaatttaaaaatatcccatccacaatgatttgatactgtagctttggaaatattatctttaaagtatttctttacaaccaagaaaatgataaatatggagcaacattagtatttaccaaatattggccacatagagtaacagaattttagaaatgaagggggaatcaatgcttatcagttagctgcctcactttacatatattaaactaaggctcagaaatttaaatgacattgcaaggttgtagaaccagaaagggaacctaggtctcttaatttttaacttcaatgcttttggctctcagtaatgttaatatcagaattcatactggctaaaatggttaccttgactttgcctaatacttaggtttcagaaaaaaataaatatgcacagataacttctgtagtttgtaaaacttggagatgtcccaggaaacagaataaaatgtttcaatatggtatcctaggaaaagaaggtttttatcccaccttataattaaatctgaaagattcctacagtgaactaagaggttcctaatgaagtactaagctgcatttagaattggagcaatgaaaggctctttgttctcctttcaaaaggttccagtacagaacaatttggctcccaatacaggcatgttttagaccacctgcaggctctaatctacgcaaatttatttggtacaaactttcttctacgtcaccttcttattcagtataattccagagaggtttattgctaacatttaacagtattaataagagaacagttgctctcatggggactgccctggaaggaaaaaagaggaaaattgagcttttgttagcttcttccctatgaatgcactgccatcaatgtgtataactttaagatgaaataatcaccttaaactgaagagtgtttgctacaactgtgattcaaacctgtccttcagacaggggtcctctgtaactctcttgattcaaaggattagcttaaaaagtatattaacatttaaacatttctcaacagaatttgatttataacaaaaacgaagtagaaatagacaagtagagatagaagagtaatttttaatttgctttaaaaaatctgatgtattaatgtattttattgactttcatagaaagaattcaaatttatttaaatttcaattgtatctactgcccttgttaggaggctttcaaatggaagaattgatataaggggtcttggcctgtgccccatacacttttgcctatgtcccagttcttcaaactttgcttggaatactgctagctccttttcacccagtgttatttaattctcattacttacatccagcccaggtaccaggtcctttaaaaaaaaaattctacctaattttccacagtttgtgtcatttgtgttgccatggtttgatgtatcaaagagaaattgcaccagacatttgttaaaaatggcaacgaaggcgttattcaagactactgtaataggggagacagattgaactctgttgaagcaaaaggtgggagaattgttcaatgctgctgtgagctagtgaaaaagtgctggaggatgttatgtgagcattggttagtatgattaagccacctgttaattggtacttactaaagttaggctcctacctcccaacagagactaggagataggcctgcctgctggcctacctttcttctttccttccatccttccttccttcatttaaatcttttttaaattttaaaatttaaattttattttgtaagactgcaaagctaattagaggctgggagaagatttatctatattccagaggggcagagaaagaatttgctattacaggttttctaaagtaaatgttctaacaagagggaggtcaggggcctatagtctagaagtctaaaatagtgtcaaactgagagaaatggcaagcccatcttggtcacaagaatatgtctgtcataaaacatagcacatgtatttcagtttttgtcttcttggcactctctcttacagagagactgtaaactcctttaaggcaagatctgagcttaattgatctgtgtttccaaagtatcctggaaagtatttgccagttagtttgagtccatctcatgaagaaattatgaatatcataatttttttttccttttgactctgcctgtgttcatttccctcagcactcattcaatttaataattgtcattctgtgtctatatggttcaaaattaattcaagataatttaatgtcctttttattcactgtgttttaaaaattaattttattctgtacagtacaagaaatttctgcagtatatcaaaataaaactgttaaaagaatgactagcccttccacaaagatgttggcagatgacctccaagtttctttgtcacccccatagaaattggctttttttctttcattattgtgctattattaagacttttcttttttttcttgctcccagagtaagtcatatctacttcattaaatagacccaaatatcccttttgatcttcctgctaacttgtactgttttttttaattgtttatttacttttgtagagacagcagtctcactttatttatttctgtagaaacaggacctcactatgtttcccaggctggtttggaacccctgacgtcaagtgatcctcttgcttcagcctcccaagtagctaagattacaggcagcatgagccaccatagccagcatgaactattaactctttaatgcagtctcaattagatcagcatctcctgtagaccactttttcgtattagttttatttttaccacttatcaatacttcatcatttacctggaaatgtggaacaaaaactagagagaaaattattcatttagtaaacttttactgaatgctttagtaaagtctaggtcttcacagatattacatatacacaaaagtaaagagaaaagtccatataccaggtgatatggtttggctgtgttcccacccaaccctcatcttgaattgtagttcccataatccccatgtgtcgtgggagggacctggtgggaggtaatcgaatcataaggggagttacccccatgctactgttctcatgacagtgagtgacttgtcacaagatttgctggttttataagaggcttttccccctttccttggcacttctctctttgtatgaccatgtgaagaaggatgtttgcttccccttccaccatgattataatttcctgaggcctccatagccctgtcagtcaattaaatctcttttctttgtaaattactcagtcttgggagttctttatagcagtgtgaaaatggactaatacagtaagttggtaccagaagtggggtgctgatataaagatacccaaaattgtggaagcaactttggaacacagtaacaggcagagattggaacagtttggagggctcagaagaagacaggaaaatgacagaaagtttggaacttcctggagattggagggtcatatggtttggctgtgtctccacccaaatctaatcttaaattcccatgtgttgtggagggacccggtgggagataattgtatcatcatggcaagtcttttctatgctgttctcgtgatagtgaataagtctcacaagatctaatggttttaaaaagaggagttcctttgcacaagttctctttttggctgctgccatccatgtaagatgtgagttgcttctccttgccttcctgtatgattgtgaggcttcctgagcaacatggaactgtgagtgagtgctcaattaaacctctttcctttgtaaattgcccagtctcaggtatgtctttgtcagctgcatggaaatggacgaatacagtaaattggtactggtagagttgggtgctgctgaaaagacaaccaagaatgtggaaacgactttggaactgggtaacaggaagaggttggaacagtttggagggctcagaagaaaaaagaaaatgtgagaaagttagtttggaattttctagagacttgttgaatggctttgacaaaaaatgctgatagtgatatgaacaatgaagtccaggctgaggtggtctcagatggagatgaggaacttgtcgagaactggagcaaatgtgactcttgttctgttttagcaaagagactggcagcattatccccctgccctagagatttgtggaactttgaacttcagagagatgatttacggtatctggtgggagaaatttctaagcagcaaagcattcaaaggcaacttgggtgctcttaaaggcattccattttataagggaagcagaacataaaaattcagaaaatttgcagtctggcaatgcaatagaaaagaaaatcccattttgtgaggataaatccaagcgagctgcagaaatttgcataagtaatgaggagccaactgttaatccccaagacaatggggaaaatgtctccagtatatgtcagaggtcttcatggcagcccctcccatcacaggcctaggggcctaggaggaaaaagtggtttaatgggctgggttcagggtccctgagcagtgtgcagcctagggacttggttccctgcatcccaaccactccagttgtcactgaaagaggccaaggtacagcttgggctgttgcttgagagggtggacgcctcaagccttggcaccttccatgtggtgttgagcctgtgagtaaacagaagtaaagaactgaggtttgggaacctctgcctatatttcagagggtgtacggaaatgcttggatgatcaggcagaagtttgctgcaggggtggggcagtcatgcagaacctctgctagggcagtgtggaagggaaatgtagggtcagagctaccacacagagttactactggggcagtgcctaatggaactgtgagaagagagccactgtcctccagaccccagaatggtagatccaccaacagctggcaccatgtgcctggaaaagccacagacactcaatgccagcccatgaaagcatccatgagggaggctgtaccctgtaaagccacaggggcagagctgcccaacactataggatgcccctctttgccttccaccatgcttgtcaggtctctccagccatgtgaaactgtaagtccaataaacttctttcttttgtaaattgcccggtcttgggtatgtctttatcagtagcatgaaaacggactaatatggaggactcagaagacaggaaaatataggaaagtttggaacttctagagacttgttgaatggcactgaccaaaatgctgatagtgatatggacaataaactccaggctgaggtggtctcagataaagatgaggaacttgttgagaactggagtaaaagtcactcttgttttgcttaccctgcagagccacaggggcaaagctgcccaagaacacctcctgcatcattttgacctggatgtgagacatggagtcaaaggagatcagttttgaactttaaggtttaatgactgccctattgaattttagacttgcatggggcctgtagcgcctttgtttcagcaaggaaactggtggcattttgctctagccctagagatctgtgaaactttgaacttgagggagatgatttagggtatctggccgaagaaatctctaagcagcaaagcactcaagaggtgactttggtgctattaaaatctctcagttttatgtattcagaaagacatggtttggaattggaacttatctttaagagggaagctgagcataaaaattcagaaaatttgcaacctgaggatgtgatagaaaagaaaaacccattttttgaggagaaactcaagtgggctgcagaaaatttgcataagtaacaaggagccaaatgttaatcaccaagacaatggagaaaatgtctccagggcatgtcagagaccttagtggcagtccctcctatcatagacccagaggcctaggcagaaaaaacgatttcacaggccaggccccagaccctctgctgtgtgcagcctagggacttggtgccgtgagtcccagctgtttcagccatggcagaaaggggccaatgtacagctcaggttgtggcttcagagggtgcaagcccgaagccttggcaacttccatatcatgttaggtctgtggtgtgcagaagacagaaattgaggtctgggaacctccacctagattaaagaggatgtatggcaatgcctggatgtcctggcagaagtttgctacaggggtggagccctcatggagaacctctgctagggcagtatggaagggaaatgtggggttggcgccaccacacagagttcctactggtgcactgcacagtggagctgtaagaagagggccattgtcctccagaccccggaatggtagatccaccaacagcttgcactgtgcacctggaaaagccacaggcagtcaatgccagcctgtgaaagcagctggaggggaggtgctgtaccctgcaaagccacaggggcaaagctgcccaaggctgtgggagcacacctcttgcatcattttgacctggatatgagacatggagtcaaaggagatcattttttaactttaaggtataacaactgcccttttgaattttagacttgcatgggacgtgtagcccctttgttttggcagatttctcccatttggaatgattgtatttacccagtgcctgtaccatcattgtatctgggaagtaactaacttgcttttgattttgcaggtttataggcagaaaggacttgccttgtctcagatgagaccttggacttggacttttgggttaatgctggaatgagttaagacattgggggacttttggaaaggcaagattgtgttttgaaatgtgaggatattagttttgggagaggtgaggggcagaataatatgatttggctatgtcaccacccaaatctcatcttgaattgtagtttacataatctccatgtgttgcgggagggacccagtgggaggtaattgaatcatgggggtggttattgtcatgctgctgttcttgttataatgagttcttatgagatctaatggttttgtaagggacttttccccctttgcttggcacttctctcttcctgccaccatgtgaattaaggacatgtttgcttccccttccaccatgattatgtttcctgaggcctccccagccctgtggaactgtgagtcaattaaacctctttcctttataaattacccagtcttgagcagttttttatagcagcatgagaatggactaatacaccagggaaattcacaatctaatgggaggatcttagctgaacttctgaactcttggctcaagtctaaaatcgtcacttagatgagggtactgagagttaaatgatattctggttgcttggaaaagaacagacctctcttctaagctgtctaccagctgcctactttacctctctactcagaggtcttccagacacttcatgttcagaatacctctattcattttctgctgatatggttttgctgtgtccccacccaaatctcattttgaattgtagctccaataattctcatgtgttgtggaaaggacccaatgggagacacttgaatcatgagggcagtttcccccatattgttctcctggtactgaataagtcatggatctgatgattttataaggggtttcctcttttgcttgactctctcattctctttttgcctgacaccatgtaagacatgccttttgccttctgccatgtttgtgagacctccccagccatgtggaactcttgagtccattaaacctcttttttctttgtaaattatccagtctcaggtatgtctttatcagcagtgtgaaattgactaacacaccctcaaatatactcctcatccaaccttccctttctcagtacatcttaccaacatactcaactgctcaggccagaaaacagggttgtctctgattcctccctcttaattccctacctctaatcagttgccaagtcctgttgattctcccatacaaatatatctcatatttattgacttttcttcattttcattacccctgtcctaatccagtcaccacctccatacaatattgtaccaactgggccatctccctccctttctgctgactttcaaagactccataagactcagtataaatgtacctgcttgttcactattttcctactagagtaaaagcctgctgctggcaagcatcatatttatcttgtctgtcagagggtcactagaactgaggacaacattttggtacatatcaggtgattcataaatagataaggaatttagaaacctcaatagtgaatgctgacattaaatagtcttctttgaaatatatttttttctgtttaaattgggtttattatttttcctcaattaaaggatgtaattctgtgaaaatttttcttaatatttgcatgttgaaaactttgaatcatttggtcaaagaaaatgtaatgtatttcttttttaaatgcatacgtgctgatgtataagatctgtacaggtttgtccaaggctccctcacagaccctatggtctagaagagaaactatttggaatatatgaaaataagatttgagtttattttttttaagtagggtactcatttattcagattaatagacagaagcaaataattttccaaagtagatgtaaaaaatgtacattcctataagcaatatattagttctaattgttaaattcttcgtctacacttgattttttatagtcattttcattttagctattccaagtaggcatagagtgctcttatgttatagttttcatttgcatttccctgtcttatgtaatatgcaactttttgtatttttaatcaatatatatttgtacatatttatgtggggcacatgatattttgctacattcatcaaatgtgtaatgattaaatcaggatatttagaatatctgtcacctcgagtatttgtcatttcttttttttttttttttttttttgagaccgagtctcagtctgtcacccagtctggagggcagtggcacattctcagctcactgcaagctcactccgtctcctgggttgaagtgattctcctgcctcagccccccaagtagctaggacaggcatatgccaccatgcccggctaatttttgtatttttagtagagatggggtttcaccatgttggccaggctggtcttaaactcctgacctcaagtggtccatccgcctcggcctcccaaagtgctgggattacaggcatgagccactgcacccggccatatttatcatttctatgtgttgggaagtcccctcttcccactattttaaagtatgcaatacattatttttaacaataatcaccttactctgctatcgaagaacttatttattctaactagtatgtttgtgctcattaatcaatatctcttcattcctcctaacccccaaccatacacccttctcagcttctgatgtctgtcattctaccctcacttccatgagatcaacttttctagctcccacataagagtgagagcatgagatatttgtctttattgctagacttatttcatttaacataatgatctccagttccattaatgatgctgcaaatgtcataattttattctttttatgtctgaatattattccattgtgtatgtataccacattttctttatccattcatcagttgatggacacttaggttgattccatatctttgctattgtgaagactgctgcaataaatatgggggtgcaattatctttttaatatactaatttcttttcctttgataaatacccagtagtgggattgctagatcatatggtagttctcttttgagattttgagaaatctgcatactgttttccatagtggctataccaacttatatacctacaaacagtgttatgagttcccttttctccacatccttgccagcatctgttgtttttctgtttttagtaatagccattctaactggggtaagatgatatctcattgtggctttgatttgcatattcctggtgattagtgatattgagcattttttcatatacctgttggccatttgtatgttttcttttcagaaatgtctattcatgtccttgcccactttttaatagaatttttttgtttttgttttttaactgttgcttgattttcttgtatattctggatattagttccttgttgtatgaacaatttgcatatattttcttccattctatagattttctcttcactatgttgattcatttgctatacagaaggttttcgtttttagtttagtcccatttgtctgtatttgtttttgttgcctgggtttttgaggtcttaatcataataatctttgcttaggtcaatgtcctatagaacttcccttatgttgtcttctaacagttttatagtttttggtcttacttaagtctttaatctatcttgagttgatttttgtatatagtgagaaatggagtctagtttcattcttctgcatgtgaatatccaattttcccagcaccacttattaaaaaagatgtcctttcttcaatatatgttcttggcacctttgtcataaatcagttggctataaatacatgcatttatttctggagtctatgttttattggtctatatgtccatttatatactaacaccatgctgttttgtttactatacccttgaagcatattttgaagtcaggtagtgtggtgcctccatctttgttcttttcgttcaggattgctttggctactcaagctcttttttggttccatagagattttggaattgtttttcctgtttttctgaataatgtattggtattttaaaaataaagattgcgggggaggagccaagatggccgaacaggaacagctccggtctacagctcccagcgtggtggatgcagaagatggtgatttctgcatttccatctgaggtaccgggttcatctctctagggagtgcgagacagtgggcgcaggtcagtgggtgcgcgcaccgtgcgcgagccaaagcagggcgaggcattgcctcacttgggaaacgcaaggggtcagggagttccctttcccagtcaaagaaaggggtgacagatggcacctggaaaatcaggtcactcccacctgaatactgcgcttttccgacgggcttaaaaaacggcgcaccatgagattatatcccgcacctggcttggagggtcctacgcccacagagtctcgctgattgctagcacagcagtctgagatcaaactgcaaggcggcagtgaggctgggggaggggcgcccgccattgcccaggcttgattaggtaaacaaagcagccaggaagctcgaactgggtggagcccaccacagctcaaggaggcctgcctgcctctgtaggctccacctctgggggcagggcacagacaaacaaaaagacagcagtaacctctgcagacttaaatgtccctgtctgacagctttgaagagagcagtggttctcccagcatgcagctggagatctgagaacaggcagactgcctcctcaagtgggtccctgacccctgacccctgagcagcctaactgggaggcaccccccagtagaggaacactgacacctcacatggcagggtactccaacagacctgcagctgagggtcctgtctgttagaaggaaaactaacaaacagaaaggacatccacaccaaaaacccatctgtacatcaccatcatcaaagaccaaaagtagataaaaccacaaagatggggaaaaaacagaacagaaaaactggaaactctaaaaagcagagcaccaatcctcctccaaaggaacgcagttcctcaccagcaacggaacaaagctagatggagaatgactttgacgagctgagagaagaaggcttcagacgatcaaattactccgagctacgggaggacattcaaaccaaaggcaaagaagttgaaaactttgaaaaaaatttagaagaatgtataactagaataaccaatacagagaagtgcttaaaggagctgatggagctgaaaaccaaggctcgagaactacgtgaagaatgcagaagcctcaggagccgatgtgatcaactggaagaaacggtatcagcgatggaagatgaaatgaatgaaatgaagcgagaagggaagtgtagagaaaaaagaataaaaagaaacgagcaaagcctccaagaaatatgggactatgtgaaaagaccaaatctacgtctgattggtgtacctgaaagtgatggggagaatggaaccaagttggaaaacatgctgcaggatattatccaggagaacttccccaatctagcaaggcaggccaacgttcagattcaggaaatacagagaacaccacaaagatactcctcaagaagagcaactccaagacacataattgtcagattcaccaaagttgaaatgaaggaaaaaatgttaagggcagccagagagaaaggtcgggttaccctcaaagggaagcccatcagactaacagcagatctctcggcagaaaccctacaagccagaagagagtgggggccaatattcaacattcttaaagaaaagaatttccaacccagaatttcatatccagccaaactaagcttcataagcgaaggagaaataaaatactttacagacaagcaaatgctgagagattttgtcaccaccaggcctgccctaaaagagctcctgaaggaagcactaaacatggaaaggaacaaccagtaccagctgctgcaaaatcatgccaagatgtaaagaccatcgagactaggaagaaactacataaactaacgagcaaaataaccagctaacatcataatgacaggatcaaattcacacataacaatattaactttaaatgtaaatggactaaatgctccaattaaaagacacagactggcaaattggataaagagtcaagaccaatcagtgtgctgtattcaggaaacccatctcacgtgcagacacacataggctcaaaataaaaggatggaggcagatctaccaagcaaatggaaaacaaaaaaaggcagggattgcaatcctagtctctgataaaacagactttaaaccaacaaagatcaaaagagacaaagaaggccattacataatggtaaagggatcaattcaacaagaagagctaactatcctaaatatatatgcacccaataaaggagcaccaagattcataaagcaagtcctgagtgacctacaaagagacttagactcccacacattaataatgggagactttaacaccccactgtcaacattagacagatcaacgagacagaaagtcaacaaggatacccaggaattgaactcagctctgcaccaagcggacctaatagacatctacagaactctccaccccaaatcaatagaatatacatttttttcagcaccacaccacacctattccaaaattgaccacataattggaagtaaagctctcctcagcaaatgtaaaaggacagaaattataacaaactatctctcagaccacagtgcaatccagctagaactcaggattaagaatctcactcaaaactgctcaactgcatggaaactgaacaacctgctcctgaatgactactgggtacataacgaaatgaaggcagaaataaagatgttctttgaaaccaaggagaacaaagacacaacataccagaatctctgggacgcattcaaagcagtgtgtagagggaaatttatagcactaaatgcccacaagagaaagcaggaaagatccaaaattgacaccctaacatcacaattaaaagaactagaaaagcaagagcaaacacattcaaaagctagcagaaggcaagaaataactaagatcagagcagaactgaaggaaatagagacacaaaaaacccttcaaaaaattaatgaatccaggagctggttttttgaaaggatcaacaaaattgatagaccactagcaagactaataaagaaaaaaagagagaagaatcaaatagacgcaataaaatatgataaaggggatatcaccaccgatcccacagaaatacgcactaccatcagagaatactacaaacacctctacgcaaataaactagaaaatctagaagaaatggataaattcctcgacacatacactctcccaagactaaaccaggaagaagttgaatctctgaatagaccaataacaggatctgaaattgtggcaataatcaatagtttaccaacaaaaaagagtccaggaccagatggattcacagctgaattctaccagaggtacaaggaggaactggtaccattccttctgaaactattccaatcaatagaaaaacagggaatcctccctaactcattttatgaggctagcatcatcctgataccgaagccaggcagagacacaacaaaaaaagataattttagaccaatatccttgatgaacattgatgcaaaaatcctcaataaaatactggcaaactgaatccagcagcacatcaaaaagcttatccacaatgatcaagtgggcttcatccctgggatgcaaggctggttcaatatacgcaaatcaataaatgtaatccagcatataaacagagccaaagacaaaaaccaaatgattatctcaatagatgcagaaaaagcctttgacaaaattcaacaacccttcatgctaaaaactctcaataaattaggtattgatgggacatatttcaaaataataagagctatctatgacaaacccacagccaatatcatactgaatgggcaaaaactggaagcattccctttgaaaactggcacaagacagggatgccctctctcaccactcctattcaacatagtgttggaagttctggccaaggcaattaggcaggagaaggaaataaagggtattcaattaggaaaagaggaagtcaaattgtccctgtttgcagatgacatgattgtatatctagaaaaccccattgtctcagcccaaaatctccttaagctgataagcaacttcagcaaagtctcaggatacaaaatcaatgtacaaaaatcacaagcattcttatacaccaacagacaaatagagagccaaatcatgagtgaactcccattcacaattgcttcaaagagaataaaatacctaggaatccaacttacaagggatgtgaaggacctcttcaaggagaactacaaaccactgctcaaggaaataaaagaggataaaaacaaatggaagaacattccatgctcatgggtaggaagaatcaatatcgtgaaaatggccatactgcccaaggtaatttacagattcaatgccatccccatcaagctaccaatgcctttcttcacagaattggaaaaaactactttaaagttcatatggaaccaaaaaagagcccgcatcgccaagtcaatcctaagccaaaagaacaaaggctggaggcatcacactacctgacttcaaactatactacaaggctagagtaaccaaaacagcatggtactggtaccaaaacagagatatacaagaatggaacagaacagagccctcagaaataacgctgcatatctacaactatctgatctttgacaaacctgagaaaaacaagcaatggggaaaggattccctatttaataaatggtgctgggaaaactggctagccatatgtagaaagctgaaactggatcccttccttacaccttatacaaaaatcaattcaagatggattaaagacttaaacgttagacctaaaaccataaaaaccctagaagaaaacctaggcattaccattcaggacataggcatgggcaaggacttcatgtctaaaacaccaaaagcaatggcaacaaaagacaaaattgacaaatgggatctaattaaactaaagagcttctgcacagcaaaagaaactaccatcagagtgaacaggcaacctacaaaatgggagaaaattttcacaacctactcatctgacaaagggctaatatccagaatctacaatgaactcatacaaatttacaagaaaaaaacaaacaaccccatcaaaaagtgggcgaaggacatgaacagacacttttcaaaagaagacatttatgcagccaaaaaacacatgaaaaaatgctcatcatcactggccatcagagaaatgcaaatcaaaaccacaatgagacaccatctcacaccatttagaatggcaatcattaaaaagtcaggaaaccacaggtactggaaaggatgtggagaaataggaacacttttacactgttggtgggactgtaaactagttcaaccattgtggaagtcagtgtggcgattcctcagggatctagaactggaaataccatttgacccagccatcccattactgggtatatacccaaaggactataaatcatgctgctataaagacacatgcacacgtatgtttattgcggcattattcacaatagcaaagacttggaaccaacccaaatgtccaacaatgatagactggattaagaaaatgtggcacatatacaccatggaatactatgcagccataaaaaatgatgagttcatgtcctttgtagggacatggatgaaattggaaaacatcattctcagtaaactattgcaagaacaaaaaaccaaagaccgcatattctcactcataggtgggaattgaacaatgagaacacatggacacaggaaggggaatatcacactggggactgttgtggggtggggggaggggggagggatagcattgggagatatacctaatgctagatgacgagttagtgggtgcagtgcaccagcatggcacatgtatacatatgtaactaacctgcacaatgtgcacatgtaccctaaaacttaaagtataataaaaataaaaataaaaaaaataaaaataaaaataaagattgcactgaatccatacattgcttgggtagaatggtcattttaacaatattaatacttcagatccatgatcatgggatgtctttgcattcgtgtgtgttgtcttcaacgtgtttcatcagatttttgtagttttccatgtagaggtctttatccatcttggttaaatttatttctaattttttatagctattgtaaaagggattgccttcttaatttctttgtcagctagttcatcattggtatatagaaatactactgatttttttgtatattgatttttgtatcctgcaactttactcaatttatttatcagatctaagagttttttgatggagtctatgtttttctagatataggattatgtcatctacaaagagggacaatttgacttcctcttttccagtttggatgcctgattgttctgagtaggacttctagtacaattttgaatagcagcagtgatagtgagcaccttgtccttttctagttcttagaggaaaggctttcaactttccacattcaatatgatattagctgtggttttattatgtgtggtgtttattatgttgaggcatgttccttcctagtatttttagtttttatcatgaagtgatgttgaattttatcaaatgctttttctgcatatattgagaggatagatgcaaaaaagcatttttatgcgaagtcacccagtactgaactttttattgttgggagtctttattgctgatttaatctgattacttattattggtctatttaggttttctatttcttcctgattcaatcttggtatatgtgtccaggaatttatccatttcatctaggttttctagtttgctaatacatacttgttcaaaatagtctctgatacttttgtatttctgtgctatcagttataatgtctcctttttcatttctgattttatttttggaggtcttctcatttttcttggttagtctagctagtggcttattcattttgtttatcttttcaaaaaaccaacttttgtttccttgacacattgtactttttaaatttttattttatttagttctcttatctttattatttcttttattctactactttgtggcctagttagttattgcttctcaagttccttgaggtgcattattaggttgcttatttgaaacatttttagttttttgttgttgatatttattgtgttaaacttccttttgactactactttgtgttgtttccattttcatttgtttcaagaaatttattgatttattccttaattatctctttgacccaaaggtcattcagagacattttttttaattttcatgtatttatacagtttccaaagtttcttttgttattgatttctagttttattcattgtggtctgagaagaaacttgatataatttagatttttaaaaaatgttttgagacttattttgtatcctagcatctggtctatcctggagaatagttccatgtgctgacaatgagtactctgcagctgttcaacaaaatgttctgtaaatatctgtgaagtccattggcctaaaatgcagtttaaatccaacgtttatttattaattttctgtctagatgagctgtctggttctgagagtgggatgttgaattcctcaactatttttgtgttgaagtgtctctctccctttagatctattaatgtttgctttgcatatatctgggtgtgatactgcttctcagccttttggctaagatcaggtttagtatctggttgctctggtattggtgcatatatgtttagaactattctatcctcttactgaattgatctcgttgtcattatataatgactgtgtcttctttttactgttttgacttaatttctgtttcatctaatataagtatagctactcttgcttttttgcttttgtttgcatgaagtatatttttctgtctttcacttctagtctatatctgtcttttctatcttttacttctagtctgtatctgtgtacaggtgagatgagcttcttagcacataattaagtcatgttctttaatcaattcacccattctatgtcttgtgaatggaaatcttagtctgtttacacccaaggtgattatcgatatctgagtcttcattcctgtcattttgttaattgatttataattgttatgtatatcttttgttcctttctctctctctctctctgtcttattgtttatcattgcagttgggtggttttctgtagtgctaacatttgattcctttcatttccttatttgtgtttgctctaccagtgggttttatactttcaagtgttttcataatgatagatatcatccttttgctttcacatgtcagcctccctgaggtatttcttgtagtgctggtctagtggtgatgaattctctgagctttgcttctgtgagaaaagactttatttttccttcatttataaaggacaacttggctgggtatatggttttttctttcagtgacttgaatatattatcccattatctcctggactgaatgatttctgctgagaaatctgttagtctgattggagtttccttataagttactagatacttttcttttgctgtttttagaactctctatgtctttgactttttttttttttttttttgagatggagtctcgctctgtcccccatgctggagtgcagtggcgtgatctcagctcactgcaagctccgcctcctgggttcacaccattctcctgcctcagcctcccgagtagctgggactacaggcacacgccaccatgcctggctaattttttgtatttttagtagagatggagtttcaccgtgttaggcaggattgtctcgatctcctgacctcgtgatctgcctgtcttggcctccgaaagtgctgggattacaggcgtgagccaccgcacccagccatctttgacttttgaaagtttggctacaatgcaccacggggaggaccttgaattttatttgtgtgagtttctctgagcttcctgcatctagatgtctaaatctctttttgttagacttgggaagttttcacctataattttgttaaacaggttttctgtcccttttattttcttttcaccttctgggacactgaaaattcaaatatgtcatgcaagctttgttcattattattattttttatgtttttagtgttcatctgactgggttatgtcaaaagacctgtcttcaaattctgattctttcttcaacttgatatagtctattgttaaaacttttgaatgtattttgtatttctttcaatgaattcttcattccagagtttgtctttggttctttgtttttatacctgtctctttagtaaatttctcattcatattatgaattgtttttctgagttgtagtctatctcactgagataggctttaatattgttattttgaattatctgggatttcaaaaaattcttattcattggaatctgttgctggaggattattttgttcatttggaggtgtcatattttcttgctttttcacatctcttgtgtctttgcagtggtatctgtgtatctggtgtagcagttgcttgttctaatttttttttcttttctttttatttgagacagagtctcgctctgtcacccaggctggagtgcagtggcaccatcttggcttactgcaacctccgcctcccgggttcaagcaattctcctgcctcagccttccaagtagctgggattacaggtgcccgccaccatgcctggctaattttttgtatttttagtagagacggggttttaccatgttagccaggatggtctcaatctccagacctcgtgatctgcctgtctcagccttccaaagtgctgagattacagccgatttattaaattatcttttgtcagggaaactttttcttgaaggtgtatctatgctgccagttgggtaaagttccttggcttttgattttgggtgcaggcagtagcatagtctctatgatttatttgactgtaaacagtatcagtagtatctatgattccttcagtggcttagagtgtggttattagtggaagcctgttgtaaaattttgctggggacagggatgtgatgtgaatcagtctttagaccccagtggtggcagcagtgggccacatgtgcctctcctaagctgctcaggacttggtggatatgtgggtcctggtgtgagctccctctctgaagcaatgccatcacatgatttccaggcagctctctgtatcagtttcagagaccacatgggttgacagacttccccaaggctaggactgcaggagtacatggtgggaatgtggatggctggattcattcacttaatctttcctcatattttagggagcctctctaggctcatagatgatgctagctgagcaggctgccttacttctctcttcttccttaccttaggcatttcctgtaatttctctgttgaattcaaaattatattttagatgataagtctgattacctactcactccattggttcttctctgtggagcaggcaagtaccagatgcctctagcctaccgtcttgaagacaccctccaagacaagtttcttaaaggaggtatgctaaggtagaaccttttttttgttagaagttataaaattccaatggaatgagtatatacaaaaagagaacattattggttcatgtaaacatgatgttggctattatcagtgcttacttgttcctgcgtttgtctctgcctcacctgcctaacagcttgttctttcagccttgctttcttcatgaagctggaactgtagccagctctaagttcacttcatgccagatgcataagcaaaggagaaaggatctccaattacacctcgcaaaccgacattcattctgtgcaaatgagaaatatctcaggggaggactcaggcccgacttgggccacacacacacccattggccagtcactgtgaaacaattttgtagactggttgtgagggcagaagtagaatttaacaaccttttctggaaccacataattgaaggaagggggaaaagttccccctaaatgattttggacgcacagttaccagaagaagagataaagataaggagaagaaaaaggaaaaaataaaaacagaaacagtatatactctaggaggtacatatggagtatcatgggaatttagaagaaagagattatttcttagaagaattcagtgaaatctgatggagtctttaaaaaatgaaggaatatatactctcaagtggttgcaaagaacatttcaggatagacaataccagcttgaaaatatgagttcagtgtagatgttttaaaaataagaaaagcagagtagttaagtgtagttaactacactttgaaattaatttttagtttattttatttttttgagatggaatctcgctctgttgcccaggctggagtgcagtggctctatctcagctcactgcaagctccgcctcctgggttcacaccattctcctgcctcaacctcctgagtagctgggactactggcgcctgccaccacgcctggttaattttttgtatttttagtagagacagggtttcaccgtgttagccaggatggtctcaatctcctgagctcgtgattcgcccgccttggcctcccaaagtgctgggattcacagacgtgagccactgcacccagcctgaaattaattttttagtccacatgaagcagatgaggaacatcaaaaccatctttaaatatatcagtcttgtagatgtttgtgaattaaattgattgacagagacagactaacaagaagttatttcagtgatctatgtggaaagtcttgagagcctgaatgtaatgagaataataccaagatataagcagaatcttgcactgaacattgccaaatattaaatactgtccctagttatttgtcagtgatattttaatgattattgccatcttcctcttttttccccattctccaaaactgcattattataatatacagtattataatatatatattatatatataataatatatatataatataatataatataataaggttatggattaactcctcttaattatctgaaaagagataacgttcatgtgctcagcacacaactaagtagtctgatttggtgttttttattttatttattttagaaaagaagactttcaaaaggagaaaaatctactgccaaataactcagcacctcttacagaaccacaagatgtggaagaaagtcattgaagaggagcaacggttggcaggcatagaaaatcaatccctggaccagacccctcagtcgcactcttcagaacagatccaggctatcaaggaagaagaagaagagaaagggaaaccaagaggcgaggagataccaacccaaaagccagaccagtgacaatggatagaatgggctgtgtttccaaacagattgacttgtcaaagactctcttcaagccagcacaacatttagacacaacactgtagaaatttgagatgggcaaatggctattgcattttgggattcttcgcattttgtgtgtatatttttacagtgaggtacattgttaaaaactttttgctcaaagaagctttcacattgcaacaccagcttctaaggattttttaaggagggaatatatatgtgtgtgtgtatataagctcccacatagatacatgtaaaacatattcacacccatgcacgcacacacatacacactgaaggccacgattgctggctccacaatttagtaacatttatattaagatatatatatagtggtcactgtgatataataaatcataaaggaaaccaaatcacaaaggagatggtgtggcttagcaaggaaacagtgcaggaaatgtaggttaccaactaagcagcttttgctcttagtactgagggatgaaagttccagagcattatttgaattctgatacatcctgccaacactgtgtgtgtgtgtgtgtgtgtgtgtgtgtgtgtgtgtgtgtgtgaaagagagacagaagggaatggtttgagagggtgcttgtgtgcatgtgtgtgcatatgtaaagagatttttgtggtttaagtaactcagaatagctgtagcaaatgactgaatacatgtgaacaaacagaaggaagttcactctggagtgtctttgggaggcagccattccaaatgccctcctccatttagcttcaataaagggccttttgctgatggagggcactcaagggctgggtgagagggccacgtgtttggtattacattactgctatgcaccacttgaaggagctctatcaccagcctcaaacccgaaagactgaggcattttccagtctacttgcctaatgaatgtataggaactgtctatgagtatggatgtcactcaactaagatcaaatcaccatttaaggggatggcattctttatacctaaacacctaagagctgaagtcaggtcttttaatcaggttagaattctaaatgatgccagagaaggcttgggaaattgtacttcagcgtgatagcctgtgtcttcttaatttgctgcaaaatatgtggtagagaaagaaaaggaaacagaaaaatcactctgggttatatagcaagagatgaaggagaatatttcaacacagggtttttgtgttgacataggaaaagcctgattcttggcaactgttgtagtttgtctttcaggggtgaaggtcccactgacaacccctgttgtggtgttccacacgctgtttgttggggtagcttccatcggcagtctggcccattgtcagtcatgcttcttctggccggggagattatagagagattgtttgaagattgggttattattgaaagtctttttttttgtttgttttgttttggtttgtttgtttatctacacttgtttatgctgtgagccaaacctctatttaaaaagttgatactcactttcaatattttatttcatattattatatatgtcatgatagttatcttgatgtaaatatgaagatttttttgtttctgtagatagtaaactctttttttaaaaaaggaaaagggaaacatttttataaagttatattttaatcaccatttttatacattgtagttctctccaagcccagtaagagaatgatgattcatttgcatggaggtcgatggacaaccaatcatctaccttttctaatttaaatgataatctgatatagttttattgccagttaaatgaggatgctgcaaagcatgttttttcactagtaacttttgctaactgaatgaattctgggtccatatctcccagatgaaaaactgttaaccaataccatattttatagttggtgtccatttctttccaacactgtttgttatgattcttccttgagtacttatatacagacctgctcattatctaaacaatcttaccttctaagtaaaccttgattgtgatttccagtttttattttctctgacgtagtagaaaggaatgtttacattaaaaatacttttgtttctcataaatggatattgtactcccccctttcaaagcattattttacaataattcatggcattttaaaaaataaggcaaagataatacgacaaaaaatatacatggtttcaaggcaaattctccaataagttggaaaatgtaaaaaggatcaagtggatgcagcctctacctaaataattaaaatatatttcagtatatttctgaattaacaccaggtcttcattatttagaacttactaaattgttttcattttcttagttttacctgtgtatctccatgtttgcaaaaattactataagtcaaattttgccagtgaatttaactatttttctttccttgcaattaaggggaaaaaagcatttatcttatcttctcataccccttgcatctaagtacttagcaaagtcaatattttcccattttccaaatgcgtccatctctaacataaatattaattgaacatagagctatgtttggagtgagtggactggcaggacagttggaagtccatcacagtctattgacagtttcatcaaagctgtatagtccaactagtggggcagcttggctactatggtggaagtctcagcaaactgcctggttttgtttgtttgttttgttttaaggtacaggaaataagaggaataatagtggccaaagcaattagaacatcttcattccagaactgtgttcagcaatccaggcagattgatacatttttctttaaaaataaattgctattacagctagacgtcaattgggataaataaagggatgaagatccactaagtttgtgactttcatacacacccagtacatctcaaaggatgctaagggacattttctgccagtagagttctccccctttttggtgacagcaatattattatgttcacatctaactccagagcttacttcctgtggtgccaatgtatttgttgcaatttactacatttttatatgagcctatttataggtgccattaaactcaggtctttcaaatgaaagagtttctagcccacttagggaaaaagataattgtttagaaaaccataaaatcaatggtaggaaaagttggaactggttacctggatgccatggttctctgttaaataaagtaagagaccaggtgtattctgagtgtcatcagtgttattttcagcatgctaataaatgtctttccggttatatatctatctaaattaacctttaaaatattggtttccttgataaaagcaccacttttgcttttgttagctgtaatattttttgtcatttagataagacctggtttggctctcaataaaagatgaagacagtagctctgtacagggatatatctatattagtcttcatctgatgaatgaagaaattttctcatattatgttcaagaaagtatttacttcctaaaaatagaattcccgattctgtctattttggttgaataccagaacaaatctttccgttgcaatcccagtaaaacgaaagaaaaggaatatcttacagactgttcatattagatgtatgtagactgttaatttgcaatttccccatatttcctgcctatcttacccagataactttctttgaaggtaaaagctgtgcaaaaggcatgagactcaggcctactctttgtttaaatgatggaaaaatataaattattttctaagtaataaaagtataaaaattatcattataaataaagtctaaagtttgaaattattaatttataaaagtgaactaattgtgtgattatcaaatcctgattaatgagaagtgaataatctaaaaatcatttatattctactgtgggaggtattcttggtgcttccatgtcagtatcgggctctccattgtcctcatcatttatagtacttctaaaataataatggaaacctcaataagggtcttgtcctcttctgtcatcctaaccctgattctagtttaatgcctttcctgacttatatgccatttgtaaataaacttcctttcaaactgttagaacgctccaataaaacaaagttctatgaactttcatagtctagaaaaaccaacaagatcccccttatttagagattattagctaatgtgtgaaagaaatatcaatagaagttcaaatgctaatagctcaattccagataattgaaagctaaaatatctatgtactttgaagccaaactgagtttatttcattgatcgaacaagcagcaacacattttatgaccattgtcctaggtcgtttgtgtctttatgaatttgctgatattgtcagtccattttttaaatcttcaacaaaattcaattttaagattaagaaatcagcaattttaggtaaagaatattccaagattaaattgttctttttaagaaatatatcagttttaggtcctatgttcacacatcaatttgttcatctggaagatattcaaattcttattgggaatgaattacactacaaacaatgagttgagctaaaaactatacataacttaaattgagatttgcatacgtatttgtgtgtttcacaccaaagacccatgctcagagaatggtaacatattttccagtctatgttatgcaccatggatgttaggcaatgtgtgctattgccaacacatggtatttgttgaaatacatgttgctttgaccttacctaatggtgcttttacctaacttgaagtgctttttgtgttcccaccagcaaatcattctaacagcaggctaacaatggtgaagtaaaatgaacatttttgggccgggcgcagtggctcacgcctgtaatcccagcactttgggaggccaaggctggtggatcacctgaggtcgggagttcgacaccagcctgaccaacacggagaaaccccgtctttactaaaaatacaaaatgattcaggtgtggtggcgcatgtctgtaatcccagctacccaggaggctgaggcagaatcgcttgaacctgggaggcagaggttatggtgagctgagattgcaccattgcactccagcctgggcaacaggagttaaattttgcctcaaaaaaaaaaaaaaaaaaaaaaagaacattttttggcaaaatatgaagacacgaaactgaagataaaggcttcatttccatagtggaaatcagtgtttacctgctgattcaaaaatactgacctagtatcagtgtaggtaagaccataaaatacagtgtatagaattatatataaatggaaaacaaaattagcttttgacatatgtttaagtctctaaaaggaatccacattttctttccagatacagctagtttttctccacatgtttttaaggttgatttccttgacacacattggtatccttgggtttctgcttccttgaaaagttgtggagcacagaagaaaagccttctctttacagataaaaaatgttgaacatgttcatgttctgacaatgaagcaatgatttcctcattgctttgcattaagaagtggttctttagttatgcaaataattcaataaccaaaaatgcttcaaagagtaataataaatcaccataggtaagtattctcaagaatgtttaaatttggtttattttcttgaagcaaagttatttaattttttaatttttaacttaaaatgaaacccacaaaatgacattccgtggtactgagtattttcacataatcagaactgtaaactactgattattttcatatactcagaggctgaatattttgatataataagaactagtatcatataacagaaaactagaaagaaggtttaattctttttctaatattctacaagcctcatggggaaaatgttttatgaaggattcaaatacattagataaaaattagtgtcaataatttaactttgtacaaattatagtctgagatcttctgagttttcctaagacaaaggagtgtattatccagcaatttattgttttgtgttgttataaaataaatgttctattaatgtatattttattttactaagttctaacacaaaaggccaaatacttatctttcctactaagaaaaattaaaccttactaatcttgactttcacagatgaaacactgaccagtcaataaacaacagccactgcattaatctgcccagcatgatcagatagtaggttagaatttgaaattcattaataaaaaattaataacaaaactttgtaagccttaatggtttaatatttccgtgtcattgaaagttcctatttcaaattaaacaaactacagcaggatatttttcctttcctgtttcaaaacaaaaaccagatgaagaaaaaacaaagacaaactcataggaggaaagcatggcttctcttcaattatacttctcttactcaatattagtaaccaaaaaataagcagaaattctcctctgccataattaaaagaaagaatggccttcgacttaagttctagttcctcaagcctgctttccttgtaaagtccatggtactaagaagcaaaaaatgcagagctgtggattgggagtgaacactacataaaatttgcagggtatcccaggacacccctcagtctttaatacaaaccaatttagttactctggagtgaaatgacttgtggatacactgttcaaccatgacttcctttatgacaaggagcacttatacatgtttccaaatgtgaattagcccttgaattagtttgaaaactgctacttgaataacttttgaagagaaagattttctttccggtaattttactccctaaaaaaggcaaaatactcagatgttcaatgtgaaattacaaatagttgtttgaagtatatatacttcaataaatagttctttgtgtatataaagtgttggttcatagcagtttccttgtcccttttctttttaaaattcccaactgattatgggtcaaaaactccatcttatatcagcgatgatttcattttaactatttataaggataatttagtattatagtattgctaactttaataattctaccatatctatatattacctgttagtgaggttttaatggctttacttaatacatatttgttcatcagcttaaaaaatcactaaattttttatactctgtaaaatatttgcatgggaattttagcttattatgtttgcttaactggtgtttttttcacttatgactgtcagtttatgtcttaatgtattctcacagaagaatgcataccatttttgttccaacaagaacaatctgttagttgataagcactggtaataaatatcactgaacccacccaccccctgatatttcttcctcaagttttctatcatgcaaatcacaaaatgaaaattggtgcttttacaaatatttgaaagctctcaatgcaaaataataaaaatgagattctccctggctttctataatatattaggatatgtgactgaaggaaatttaatctgaatttagagtgttttaggttttggttggtttgaataatttgatttgcattttggtattaaaatgtgctctgatttctgcgggcatttgttctgaccagtcattacctcttcccaacagaaaaaaaaaaaaaaaaaaaaaaactggcattggcaagttttaattaatatggcccaaaaatttgaccttgaaggatatttgtaaaccagagagtatttaattaactaaatttactgtttctgaaaagcaatattttcaaataactgccaaccaaagtgacactatagggatgtcatttgtacatgacaatttttaacaatgtaattcttaccactctcgacagacctgaatatgtttactaataactttgccaaatttttcaacttctagaaaaacaagagtgagtcttttgaacttcatcattttgcaattgaaataaatcttgtcctttcacctatatcttccatttaatatatgtagcaatacttagtatcatcccatcacaagtattgttaggcgacttagtggcggagaaatgtttgtaaaaagtagaatctgtacagaaaaaaaactagatcaattgtatttatttttattattttttagcttagatactatgttgatgctccctttttgccagaattactggaagtgcctcttggttttatatatataatatataaatattacagtaaatatatactcaaaattattaaaattatgtcctagattatctatgtcgatgttatgagtgaagataatgtataatataaaaataattctgcagtattatgatttgtgtagtatttgtacaatccacttcctccagtcctctgaaggcatcctttcctcttatatgcaaataccatggtgtggtacctaacttactctgttgtgagacgacatctattggccttcctacaatatccaaagaaccttgtggggcttatattctcgtggtcacatctttctcactatcactaatgaattcatagccacatttagtcatcatctaactttctgcaactaagttgtgtcctttctttaatcaaatagcaccaaagccaaaatacccacatttctggcattcagctttagagtcctataaaatagacagttgtatttctacctaggtttcctaagaaattgattcttcaatctacacctattatttccccattacctatgatatcaaatcccaactcctaagcctgatgttttcaaatccccctttgctctggcctgaaactaccctctccagcttcctgctccaaaaggaaccttccacttatcatttatatttctttatctcaggcaaatacatcagttgagttagtgcactttctataaacatacactttctctctctctcacacacacacacacacacacacacacacacttccaactatacacagcacaagaagacgagattgactatctcttcttcaggtgtgaattttactcctctctcagggttttcctcttacaatcatgtcatggctctgagtggacgggagatgattgcttggcagtctagtctgaacctttaacgccatccttcaattaagtgttcttaaaacaagtcttaaggaaggaaaatttaaattacatggtgtgagggataataatgggtaagttctaatggagactaacatccaggccagagaagagggattattctgatcattacctaaaataggaagcctgagacatcagattagtgtgtccttgttcacaccaattcacacgtagtccagctttacaggtcacccccaagcctgtaaattctaccccatgtggttcagcaggctttgggggtgctttctatctctaaaatttttggttcgacttgttcagatgaaataatttccagtaagggatactttcaccagaatacacagtggttccattaccctggagccaaggctactatttggatatttttttatactttaagttttagggtacatgtgcacaacgtgcaggtttgttacatatgtatacatgtgccattttggtttgctgcacccgttaactcgtcatttaacattaggtatattttgttgtctcatgttactgaaccatcagggtagaaagtgattgatcccagtttactaggctgaagtgtggtttctgatatcaaaacagtggcaagaaagattgtgtttggaatgcaaagaattctgcatggagcccttaatgcttccatgttacatagtgagtgtggtgggacactggagtcacccaataaaaacagaaccactaagaaaattgaccttgcaagaattaacttgcaggtcatcctatcagtaaaaatgttccaccatgagaagtcctaacagagggcaatgaaaacgtggaatggatagtggaaaacaaaagttttatgtcaacattggcttcgtagtcaaatatagaaataaatttggaatttatccactaacactttttaatctatttacatatgtgtatatctgtctctgtatcatttatttaaaactcaatatgaaaattttcaaatgtattcaaacaagagagaatagtagagtgaacaccaggtgcctatcacctagctcaaacaattaatatactgccaatctgttccatacatacctcccaatatttagtacatctcctctatatgaaaatatatgaaattctatatgaatatatgaatttctatatgaaaatatagaaatatagaaatatatgaatttctatatgaaaatataacatatccttctattagtaaatgatttgtaaaaaaaaaaaagaaaagaagtgctatatttacctctaaaacaattataccaccattatcatccctaaaaaaaactaacacaaattactcaatacaacaaatatccacagtgttctcatttctgaggttataaaacttctaagaagattacaaagaagtcttcttgttatagtttgtttctttttgaataggtgtacgaagtctgtacattgtaattggtcagttcatttcttaagttaaaatatacaatttctccctcctctccttttttccttgtggtttttggttggaaaaaatattgacttatttgtcctaaatagattctttactttcaattttgctgattgtatcttttgtggtgtcatttaacttgctcttctcttccctatatttcttatggactggtacttaaatctaatctgagatttgataagattagttttctttggaagtaaaaaggaggactatttcaaaggtggtgtttcaagcttttaacttccatcaggatgttgaaaatggcttgagatttttggtgatattattggtcattgattacctttcgctaaattcattaattcattagaggtttgaaaattataatactctcacttataagtgaggctaaacattaggtacacatggacataaaaatgggaacaatagacatggcacattacttgagggggagatggaggagaaaggcaagggctgaaaaactacctattgggtaccatgctcactatctagttgatggaatcattttaccccaaaactcagtgtctcacaatatacccatgtaaaaaatctgcacatatactctctgaatctaaaattaaaaattataaaattaaatatctaatagcttaattctactagaaattaatctacttatttataatatagaattataaaaactttatagcgagggtctgaactgatcatattaacagaaatttttcaaaatggttaggaatcatccttatacaataactactttctcaatttctctactgtgccacaaaaaccaagctatgcaaataaaatccattagagctctaccaatctaaaagcaattgaaaattagtatattttctacatttttttctttaacctttgcatttgactcaaatgagtttaaggattttgtatttttaatattgtttttattaaatctgttctagaaataaatttatcatctctggagaatagtctctattttcagactatttgttggactaaaccaaggttgcactggtattatttcagtaaaacttcaaaaccttaccaaaatcctttcaggatactttaagattgaattacatttcaataaatattaattaccctaagaaagaaaagaaaattataatattcttattctctcatccttttttgtttattagttggaattctatgaagagaaatttgtctttactctgtaataactctaaggaaagaatggaatagaaaagctaggataaacacacaatttctccctaaataccactagttttaaatctattaatatttaagttgtgtttagaattactccaaagtgaccatgtgatgttttaatagtgtttttaactcataaattttgtgattttatatgtttcaaccattactattttatatgtttcaaccattacaattattcttaccaatgctcaaactttcctatatttgattagcagaaacctgttcaggctggcttctgaatcctattggcacaatgtagtcttcaaggcatcccagctcttttttatgacaagatattccaggcctatcttatatctttacacctggtattagtcacatatcaaggatccatggtgcttttatttatggtgctagttgcacttattactacttagttgatcattgtttctagaccttttcagtggagagagctaatacacacacacgtagatatctatatctatctatatctatatctgtatctacatatctcctgtatatatgtatacatttttccagactgaaaaactcaactcaaatttgggtatacaaaatattccctaacctcattggatcttatattatagattatatttaaagccatgacaccagataaggtcattaaggaagtaaatatagacagcaaagaagatattccacagactgaattttggaatattctagcaatgaagactaagagcaaatgaccactgaagtaggaggaaaaccaggggtgtgtggtgaaaccaagtgaataaaatgtatcaaagagaagggcacattaatgacatacaattctgctaatacatcaattaaaatgaggtctggatattgatcactggatttattagtgtgaatgcctttaatgttcttgatgaaaatatttgcattggagtggtaaaggcaaaacctgattagagttggtgaaaaatgaactggaggagaggaaatatatacaaggaatgtgactaaagtttgggcttagaaacatggactgtaggagcaagaaggaagacagctaatagcaatgtacatttgggtagagagtagatgtcatgaggacagagtataatatttctcctgatatttctgtttagttagtgaatttcctagacaagatcatcagctaataatcagggtgcagccagaaatgaagatttgagagaggtaaagtgattgggtcattaaaaagtattacgtctagcaatgcctctatgactcagtgagtttagtggttataaatataaaataagccctaagggcatgttactgagttgttcttcaatcacttaaaacagtgcggatgcaaatactaagaagaaagaaagtggatttatctgaggtgagctttgcctgggaagatcaataaattgaaagagaaacaggagaattgagggtatacagaaggaagtgattaaaataattgcatgaaaaaattcaagtaaggaggaaaatgaaaatatgagtgtactggcttgaattgtgtctcccaaaaagatatgaggaagtcctaacccccggtacctgtgaatgtcacctttcctgaaaagagagtcttagcagaccttatcaagttaagatgaggtcatgagggtggtccccagccaaagatgactagcgtttttttaagtacagaggacagagacataaagggagaagatggccatgtggtaacagaagcagctgttggagtgatacacctataaaacaaggagttcaggctgtcagcaaacaccaggaggtggaagaggtaaggaggattcttccctacaggtctcagagggagcatggctctgctgacaccttgatttttgacttctggcctccaaaagtgtgagacaataaagttctgttttaggtcactcagcttgtagtactttgttagtgcagtcttaggaaatgcattcatttatatgttgtctgtggttaatttaccaccagaatggcagaggtgagtggttgtgatagagattgcatggtcctacaaaacctaaaatatttcttatttgtccctttatagaagtttgttaactccctcatctagagtttggaaatgggaatgaaaggctgaagtaggctacaatccaatgcaaggttgcacataagaagctctgaataagtgtaaacattatgtgcattcaaaacagtaacattaactgtggactctggaaataagaaaaggaatcgtgttggagagtgagactgagccagttgggaatagacaaaattacattctcaagggagaatttcatttcttggtgaggaaaattatgccctttgggctggtaaatactgtctcctcttgaataaaacagtggtctggccacctgttagacactgagaagtctagactaacactcaatgtattcggatgtttgctcactatttccgatggaaaagtctggtttgattgttaaagtaaaatatttggcctttcagagtgtgaaaacaccattgcttcattgcaaggtttaatataaacccatttttcttgcattttcttttctttcttttgtagtaaaatgtgcttgaataatattaactattctgaccattatgtgaactaaatatatatatatataaaatgatgataccttccgctcatttgcaatgtgcttttgaatacattatataatttaatccttaaaacagccctataaatctgttttttttttttttaaacaaaaaaattctgctttacaaaggaagaagttgagactcagtaaaattaagtgaccatcccagagtctcagccacttaagagtagggtgaatcttggataattctggtagcaaatttatttattggtagaaaataaatgcaaactactactgcttaagatcctctaaagtttttctcttggaaaaagaatgtattagaattgtggactttctcagtgcaaggatttatttatccttggaattaggcagaataaacaattgaatgccaaaaattcaacgtgtttcttttctgttttcaaaactcatagaaattgtaaatatgagcaactgatcatgtctctgagaattaactctttttgtacagagcgctgcagagctttgattgtgacctgatacttggtccttaaagattctagaacaccaatggaaaagaaaaccatgtagcaaattagcccaagaaccgttaaagacaaaacaaaacaaaccaaaaccaaaacacacaaacaaagaaaacacaaggatcccagtgatgtccctaaggaatagttggcttaaaaactttctggaaggaggaggggtctctgttataaacttccgccctcctctggcttctgttccttaccctctgccccctgcggagttgtgttttctgggaatcagcaagtaagatcacaagtggtcttttcttttattcactctctcccataaagaaagctttatcacgtgtggccttaaacttgcagcaaattgcaaagaaatggctcaaaagcttcagctctttctgtgccctgggagctgagatgcacgtcagtggccttgccagcgtggccaattctctgctgactgccagaaaaaagaggccaggaagaaagaggaaagagaagagatcgctcaggggtgagaccatgcccttcatcttttcttttccctaatctcctctgcttgtgtccacccacactctccccacctggcaaaattgttcaaaattgctgtggagtttacctcagtttcctctttcagtctgtggtgtgtggtccatcctcttgctgagcacattgaaaggaactggctatctttgatctcttcctccaggtaagacttggttctctcattttatattttgtgactaatacatcagattcttatgggagattaacccttaatttctgtgcgatttatgttttatccataaaaatatcagtgaaaggtgatgatcccctcattagtttgatggaaataagagagaagattggttaaagaaatcagataaactcaaatttgagttcaaattttgagtctcatgcttataaattgtttagcttttgacaagtcacttaattgctttgggccatctaatttaaaaaatggaattctaatattacgtttgttgggttgttttaaggattagaaaaagtacgtacattactcaacacaaaaatagtacttattaaatctgaatctgacacagataatttgtattaggtgagaaattagtttctaaacaacacacactcctctgcagcaagtgccgtgaggctattttttagtttgtctgaagggaggcatcctggtgtccagaagtcaagccaggacagaggttttcctcacaggagatggccaagagaagagagggaggagcgtggttgcctttaggactttctctgtggcagtccttggggactgattgctggacagagtccgagtcagttgcagagcagaggggttgagtacatctgcgggatctaactacaaccttcctaagccacttgctctctccatttaaaatctggagggctgattttgaaactaatgaggactgattattcaatgtttttgaaccaaaggaagagaaactgcttggcttgtttttctttggcctccagaataagttatgctccaaacccagggtattggatgaaaactgtcgtatgtcttttcctggaagaatctattcaagtggccagagatcaattaaggatttgccaaatgcaaataattctgaataactgtgtactcttgctctacttctgcagttgcaaatgaagagagagagcctgcaggccccaaggatctttccgttcatgtcattgagcttccttccctctctttttacaggggatgaactgtagcctccttggaacatactacacttgcaataatttgcgcattaaccatcaagacacttcagtaaagctatgcagtgccaatagtttaagctggatcctattttgccctaacacaaagtgaactgagatgactatatttatatttcattatacttttgaattttagttacagtgatttttgtgtcaacacctccatatattagataacttaaaatgtgctatagtatctcaagtttttcagtagtgtttttaacataattattttttctctttgacattagcattctagatgtaacactttggatcttttaaagataaatttaggataaatgatagcagattttattattaaacaatttatttgtattttctggtcactgactttaatattatgactgatgagaaataatatgatatctgaatccccaaaaacagaaggccaatttgaattatataatttaatcctttgaagaatattgcaatttaagccagaaatttaataattatccgtttcttattttaaaacctttctggtttgtatatctcttgcttggcaatatttttactcatagttttgtagtaaagatacctatttaacattgtataactctgcatttcccaaaccctgttttccttgtcctggcaaaatactgttctagaaaagtagtcctagtgactcagtagatataggaagttggctgttggtatcagatactgttaaggaagaatctctatattcttcatttcttaaactttgttttttaaatcagtaatagatgtcgaattttatcaactgatgtttttcatgtaatgaaattctattttttattttgaattattcatgtggtaatttaggtgaataaatttcctgtaattaaccaattgttgcatcttttagaataattctaatcatagagcaagagttaatttggatttgctagcatttacttagaatttttataatctatgggcttgatctgaagttttcctttctgtgctatctttgtgctgttttgttaccaagataatgttatttttgtcaaactaatggagacaatgattatctgtttccattatcggctatctttcctcaaagaatatcccttgataaagcacttctggtgcatcatctatccttagtatctttgtttagggaatagtttcagagtatttttgataatgtctttcttggctattttctcttctggtttttatcaatataggtaatgaatatttgctagaaacttatccattaagtcaagatattaaacttacttcaacattcgatgcatctttttatgttttatactcctaaatatctgtgttttatctcattaatattatgtatttatatctttgttttttgtcttgattaacattattagctctttgttttgcttattttttgtttttaaatttttctctttcactcactcttctttttcaattaaaaaattaaagttatgaatgcttataaaatgaatccaaaagcctaataataatttttttgaaaccagacccttgtctcatcctttcctttagtcaattcccactccctagaggaatccacttaatttttaaattgttgaagtcctctgatgttttaacttcatattattaatacaaagcaaatcgtttatactattacttgatttttcagtttgagaaagtatgaattgaaatggtgagggatgaggtttagtgaacacaaaacaagcattgcataaagtcaaggtgatatactcagtcagaatagatttctgccacaatacatgaaatttgactcctaacattattcctttaggaataacattattccactcctattatgtggatatagaagtcctctattaatgtacaattgggtcatctgataatattagggagattgagaaatactttgtttgttgaattccttgatttaggttagattatcaggcaggcactgagccatgtggaatcctaaagaagtaggggcacaggatatgtaaatattgatgtgaacagggagaaaataggttcagtgtgtgatcctgtcctgtgaggggcggtgggaaattgagcatgggaagcaaggctgtcttttttactttaaggctgatcaaagactgttctttcacctgaattaaatctaaggcacaaatcatgttgctcccttatttatcaaacaaaaacaatacacaaattggacctcttcttttttgtcatattttaaggctcacattgggtgctacaggaatcataaggtggtacagggacctataatgaatgtaaaacttctatttgttttgctaagaagatagatttaaaggctttaaagactttatcctaatgaatcaatattcaaatagttgaattcctcttcttcctttcctgcttgctcctagtttaaactggcaagaccacaaagttagcaaaattctgcctcattatttattccaaaggcaacacttaaatttattctttgccataatgaggcttaaactgggcagattgagaaagaatgctggctacagagttccaatagcaggattagttaatcagatatcctggttgattttaattgaaaacatttgccaacttactgtgacaacaaaaggtagattcactttaaattgcgtctatagaattgtggtatactgttgaataaaaaaatttaataaattgcgtagtatttatttatttttactttaaaaactaactttgacagatcagagtcaaggaatgtgtttataatggacacttcatccaaagaaaatatccagttgttctgcaaaacttcagtgcaacctgttggaaggccttcttttaaaacagaatatccctcctcagaagaaaagcaaccatgctgtggtgaactaaaggtagagcaaccaagaagtaaatagtgttgatgctcttagttttctgtccagatatcatctatataatgaagttagaatgagaattgttttctcctttaaaaaaaaaaaaagatcttagatgcaatttactaaaaccacaccttgccaggcggtgcaagcaatctctacctgaccatgtgggttaaattactttcctgggctcatttaaatgcccccccaaacagaaaagagagaaatagtagaaactagtatttattgaacttctgccatgtgccagatattaagccaagcacagatataacatttaatttgtgaaacatcacattactcccattttgcaaataaaaatgggaataatgttacagagtagggagaagagttgcttttttttttttttaggattgttgtatcaaacaatagtttttagttttttgaggaacctccatactattatattttccaaagagactacatcaacttatgtaaccactaaccatgtacaaagattcccttttctttacattctaaccagtttttattttttgttatagccaacagatgtgaagtgatatgtcataaagatttttatttgcatctccataatgactagtgatgttgagcacctttttcatatacctgttggccatgccttcttttaagtaatgtctatttagattatttgcccattgttaaaatcatgctatttttctttgctatttagtgatagaagtttcttttttttaattattttttaattattatactttaagttctagggtacatgtgcacacgtgcaggtttgttacatacgtatacatgtgccatgttggtgtgctgcacccgttaactcgtcatttacattaggtatatctcctaatgctgtccctccccgctcccccaatcccccgagaatagttgtttcttaagggatgctttgtgtgctcctattctttctttatacaaatattcactcaacaaatctctgcttttttcatataaatgctaaccttccctatgaatgttctatcacagacccatgtctatcaaaatatataagctagatatcaaagacatgttttccattgagaaaaatatttgactatgaatttaataagtataaacataaacacaaggaataatagttgcactttttcagtctgtacattatgttcccggctggtgcatgcaccaccctgtgaaatacataatcccaagtgctggacttgttataggatgtcaagtgtttggcttatggtttcatttactttcttaccaatgaatgtctcaccatacactgactcttcaatgtaagactcctttgagtgcccctctggagcagagtggaagaagacaaaagataattcaatacacatttcgcataaaaaccaaatatatattttaaattccaagagaatttgaactgttgaaaacttacagtgcttaaggctgatggaaacctcgaacagtgtttgaattaaagtttcagtgataataaagttgatattatacattgttctttgttgtttgttttgtatttgtttgtctatttacttagtttccaattgtgtgtcaagctggagtcagactaagtgtgacctgtcatattttccaggtgttcttgtgtgccttgtcttttgtttactttgccaaagcattggcagaaggctatctgaagagcaccatcactcagatagagagaaggtttgatatcccttcttcactggtgggagttattgatggtagttttgaaattggtaggtattacagatgcctgactttaattttaagcccaagatcttctaggtgtggctaaagaacaccttctgcattctattgtctgctgggatcagactctattcataaaatctttttttttttttttggacagaatttaccatgatcttattctacaggagaggcaattttctcagagcaatcaacagtaaatttttctccccaggtgtttggtattgtatggttcacacaattcagagttctacatcagagtaagtgactttcttggaggaaagggtgagatgctaattggtattttgagttataaatatgtagaagtatctttacagaagaacaatacaccctgggagcactatgattcaattttagaggtgtggaataatccaaatgcgctctacaagaatttactgcaatgatgaatagaagttacatcataaaagggatttttccacccagtggtctagaaagaagaataaagggattctattaccaagaacacaagatcagagcaggcaataaaaattcaaatctcaggtgcctttggttagcaattgactacagactccttagaaattaaaaagatcgagtaatttgagtcactgttttagaagtaatttaaagaggataatcctagttagtctgcttcctgaattgataaactaaaaggctttaattttttgctgaagtaaacgaaatagtaccctctctggtccttaaggcttgtttagattcagatatacaccatagtaaaacctctaactgctccaattttagctttacctggtgaactttcagaaaagtgaataaatatttttcaactctttaacaatttatttcttttcatacatactatgtgttttggttcttactctgaatatcacagaaaaaataaaatgaataagtaaccttttgccttcaggcatttgcttcccaaattcatcttttgtcatccttccagaatgacatttcttagtgccaaattttaatgtccacacaaataactgggcaatcttttaaaaatctttttttaaaacctgaattggtaggaacaggataaaattctgcatttctaataagctcctaaatgatactgatgcttctagtcagtagatcacattttgtgtagcaggtttccaaaacatctgatcacatcaatcttttatgtaatttgcttagattggcccggatggttgatttattccttgagccctgttctactgatggccctgcactgagtgaagggccaaagagaatcaaggtaaagggtgatacattatatcatgagagaaaaacaaggagaaagaggtaactggtgcagataaaagaatagagcagatatgagcaggcaaaacattatatatcatgatgttttgatgtttttctcactctagattattatataaaaatattttagagctgaagaggcatagaaatgatttggtccaaagttttgtttgggctcccaggtcctgatgcaacttgagagacaaagtttatatttgatagagagagtatactaagttttcacaggaagtagcagaaaaccatgactccaaattcacattaattcttccagaccattctaggtatataaattatttattggattggatgccttaaaatggcttaaaagttctcttacttccattgaaaacctttatcccaaacatcacaggtttgccctaattatttgagttacataaatttccagacaaatgacttcgctgaacataatcttaatttacttcttttgcctctttttgcattccttcttttcaagattctagaacaaatatcatcctccgtctttatgttatttgtatgtggcaactttgttgaaattctattagttttaattgattttccatggaaactgtagagttttgtggacaaattgtctaaaacaaaatataatttcgtatctttctttgcaactcttagatttttgttgttgttgttgatattaccttattacattagcctcagaactgtgttgaagagaagcatttacaatggacatctttctcttgtgtttgaccttgatgcaaatacttttaaaattttagtattaagcaatatgcttgctttgtggttttgatagatacactttaaaagctggtttgctcagatttgttttttcctaacataaatcaatggttaattttatttagtacttttacagtgtctattaagttaaccatggaattattccccttccatttgttatatagggatttaattaataatttattgttagttggtggtactcttgtactactataatgaactgcgcttggttgggatatttttatttgtatatactattccgtttgctaataatttgtttaggatttttacatctatcttcataatcaagtttgggctctaattgtcttttcttatattgtccttgtccagttttggttttaagaaaatagtagtcttacaagatgaattgagtagctttcttttttttctgttatctcgagtgtgtctgtgtgtgtgtgtgtgtgtgtgtgtgtgtgtgcatagacagacagaaaaagagaggagagcaagaatatgttactttaaagtttggtaagacatatttctaaaactttctgtacctgtttttttgtgagaggctaatgatggtagatatggaataatttttatatccaatttcattttttaaagattttgtatcatcatttatatttcaaatgtatctcctgacaactcagaaaggatactaaggaagcaaaaactttgatagatacagtatatatatggtatatagatatatatatatatctgtattacaggtactgaactcatattgtcttgtatatgacatgcccattaacgtaacacagaaatagcttgtaaacagaagtgtttattgtgtggtattggtggagtgtaggcgagaataggagagaattacctataaagtgaatgtcaactttcattttatgttggttcgtttattgtggtacattaacaaacatatagatagaattatcaaaataatatgatacacaatgtcttctactaaatacttaaatttcaaatgagacatcttgtgctaaattattcgttttttaatatgaacattaagaagagcttaattcttcttggagccataaagactaggaccactgtgcaagtcatgtttctcactatcattcttattatcctcaagcccacaaaaattactgctttatttccagtttactaaagctggcaatgtgcgtgaacagtagtaaattagatgattgcttacagatgtagggaaacatgctttgctcaaaaattggaaatgaggagaacttcaaagcaaggccacccattagccatcacaatcaattgctgggaaattgttaagatcttactgtatttagcatggtacatcttaaaaactaaatatctcaaataaatatagctaaagctttaaagccccttgaagtctaaaaccctaaacaatgtttatcaaagcatgttctaggaatcatctccatcaacacacttgagttagtattacaaatgcagatttcttggcctgacctcagatccatggaatcagaaacgtttggtacttgccaagaatcgcatttttaacaagttttcatttccttaaccgtttcctgtcagtatttttacgcacagcaaagcgtgagaaatattacttgaaacatgtttaatcacttatcaccatcatcaactaaaagtaacgtgtcacatgcttcattagagacattttagaaaaccataatttctactttcaaaaaaacccaatatttttaaaccatttttcacaaaagagataaatgggcacattttatagagattttgtatttcataaaatttgctttataggcatatgttgaggttatataattgatgtacacctgaagtcgcctatgtggaataatttttatttctattttgagtcttaatctttttgtacaaagtttattaacaaatcataaagtatcatttgtgtacatgtgtttcatcattttatttcatttaaagtacagttgtgtttaatttttctataaaagatatcagctaaataatttgtaatgtgtagtccatgagcaattagaacactaatagaagttttcacataaattttaatgaataaagaaaaaataagaagtttacttcaaatttactaaaaaatatttgagagggaagctaaagggagccttctagaatttgacttactgataaaatattttacaaggacaaataattaggttgggtgaagtgaggtagaaggattgaattaaaatatttcgataaagggataaggtgattgaagtaatgaacatgagaatttcaagggtcatctcagattaaaggaaaaaaattaaatgatgcagagatggcttagttaaaagaaaacattgcttggtctgctgtatacattcattcagttatgctgttgacttctttctaagttctctaatttatgatgttttattcttttcctcaaagggaatctcttagttataacatttgttagctactttggagccaaacttcacaggccaaaaataattggagcagggtgtgtaatcatgggagttggaacactgctcattgcaatgcctcagttcttcatggagcagtaagtctaaagcaatcatcttttccttgcctttccaaacaactgcatttctcccttttatgatgattattaattatgcaaatttaagcttaacccatgataactttgtcttacttttattacattctctttggataaaatttcacaacaattttataatttttcttctgacaataaactgtaaaaaattgatagattactgtatattgatatcaacaagattaaagaaagattttacctacatgttttgagtcaaatgctttaatttagctgtaggggcaaaatatataattgaaaataaattaacatggtaaatgtcaaataggtaattaataaacactgcagacaataaattctggattatggaacactggctgctagacaattaactcagggatatattatcaactcaaacaagtattcaatgtccatacagtatctcttagacctttatcaatggaagcagggtgtctgtatcctctaaccctaaagtctacttttggtacttttgttaatacatgcacatgactttagtagttccaaagatttttacctatgtcatttttacccttatagcaacctgtggggtagaagaacatagtttatcttttttactttagagctaaggacaggaccccatgacttgaaaagtttgatgacttttgtaaatttaggtgtcttgtaattatggaatcagaacctaaaacaggtcagctgacttctcaggcagcagttttcatacatatccatgtcactgcccttctctgtgtttatctattgctgataaaagaaatgactaccaaaaggttttggagtaagagaatattttagaagattatatttcaagtgatactttattgtccactctagatcattgtatcaggacagtttagagctggagagacttagaaatgatctagtctgaactttttcttgaatttctagaccacttatgtaagtacagtcagatttttgacctccctggccaatctatcaaggtagggctcagaattctctgagtgggagcttttctgcccatttggatgaattcccaggaccctaaatttctacttggagggagttctgtggtttgggcacttctgcctgctctttggacaacatcctttcataggtcagaagtttccttgatcattgagattctacaaaaatgttatgtatgttcagcctgtagaaagaatgggaaggagaagaatgagaaaatgggtctccaacctgcctactccctgcttgatctttagttgtgttgctgttatctgttgattcattgagcttccatgtgacagtttgctcagggggaaaagcagttccactactgaaaaaattaaaaatgtgaaagtagtcacataggatagataaatccattacaccagacttaaatcactctgcagtggatcagtagctcttctatgtgacaatggctacactacttaatttttctaggtctcagtttctgcatcttaaaacaggtattaaaaataattcctagctgataatgccattgcaaatattaagtgagataacacaggtaaaatgcttatcaaggttaccaacctaactagtagctatgtcatcattgttgtgtgttatcaatccttttagctaattaagccacctggttttaaccagcaatggttttgtcttaaagctgctgtatacagatgaataattttattactttaagttagtgacaaatatcgaatacccgtataagaaaatcagagaaaaaaaaatgtattaaaaagaaggtcggacaaccagacaggctagtgttgacaaaggatatcaaagggacagagacagattcttatccaacacttttgacatggtagaggatatatttgtttatttattatttaatatatttgccaggtacaatgccaggtaaaggagctatttctgcgagaaaacaaaatagaacaaaaacacacacaattcttccttcatgaagcttgctctctactgaggattatcactctagtttctttccacctttacacaacattagtaactaattgggtacttatccaaaatatgattttcgttctctaagagataactatgctctgtctcctttgcagaaatatccttaatgtccgacaaaagctattaatcatggatagcaactattgagggcctattcctaataggctgtttagttttatgtattagctcatttaactcattaatttagcaagtgtatatatatttgtgatatatgtatatatatataatttttctaggcagtaaagaaagagtagtgacaaaataatctctgccctcacagatcttctgaggaggagacagaaaataaacagataaatacagtatgtcagagaatggtaagaactatagagaaaaataacccagtaaagacacataaggagtatgtgtggtgggttgggggtggttgcagtatcaagtagggtagtcaacgtatggctcacatagttggtgacatttgaagaagacctgataatagtaaagaggacagtcatgtggatatctgggaaagaagacttctagcctaagtgaacagtgaaggcaaagtttcttcagcaggaggaagtctggtgcatggatgacctgcaaggaggcctgtgtggctaaaatgcaatgaccacggagaaagcacagaagatgaatgctgagaagtaaaagggaatgagagaatatgtcaacgttgtttctcactttgatgggcatttgaatcacccgaggctcttagtaaactacagattgtgattcagtggccctggggttggttctgagattcagctgatgttgctggtctgcagaccatcctttgagtcataaagttaggacaattacaagaatttagtttgtcctctgaatgagatgcgaagtttttagaaagctttaagcagagtgacatgatcttacttatatttttaatggattacactgactgctgcttggagaattgattctggtgggaaagcttggaagcagggagactatttaggaggctactgcaaaatttcatacatcaaagtcaagagtgcatcagtcaggatttgaatgcgggtcagaattaaaacccctaatataaaagcatttataatattagtccccgatttaaataattacacttttgtctcttctccaacagagtctcaataggtacagaccacatccatagctctcattttacaactcagttaattaaaacctaaacgttaatttttttagagtatcacagatagcaattagtaacaaaagtggctcttagataagctgccagttatttgaaatcaaaataattatgatgcttgcaacagtaacctgttgacagaaggaactgagttaaaataatgaaaaattttcaaaatactccatgatcaccatccatacttacttcaatagtcttgaagcatcataacagtcaatggtttctctgtaattattaacaatataaaattatatttaccatgatttcaatgctaaatgttctcatgtgtagttaagaattgaaaacaggccgggcgcggtggctcacgcctgtaatcccagcactttgggaggccgaggcgggtggatcatgaggtcaggagatcgagaccatcctggctaacaaggtgaaaccccgtctctactaaaaatacaaaaaattagccgggcgcggtggcgggcgcctgtagtcccagctactcgggaggctgaggcaggagaatggcgtgaacccgggaagcggagcttgcagtgagccgagattgcgccactgcagtccgcagtccgacctgggcgacagagcgagactccgtctcaaaaaaaaaaaaaaaaaaaaaaaaaaaaaaaaaaaaaagaattgaaaacagttaacctcattaagtgattcttaacctactatggtcaaggaaccctttgataaaattataaaacctgtaaccctcttctcttaagcattcacataaaaacatacatacaaaaatgttcttactttttcagtggggttcatgaacccattgaggtgcagattgcacaaaacagatttaaggtaacctgtttatcaacttgaatatttcagtaatgcaatacatcttttccttaccatgctgaataaacagttctctacttttctttttttttttttttttttttttttgagatggagtctcgctctgtcgcccaggctggagtgcaatggcgcgatcttggctcactgcaagctccgcctcccgggttcacgccattctcctgcctcagcctcccaagtagctgggagttctttactattaatatgcatgccaatgcatattcctgtgattactagcctttggccaaatagtgcctgaataaaatgagaatcaagatgatattgagatgttttcttaccagatacctcttccccaaggggtacaaagagtgtggagaacgtggcatatgaaggataatttgagtgcaagagtagagccgggggattgtagattagcattatcacaggctgcccactaagcaatggctctttatcccagttgctatcataaccttatacacaggggaaagggtggaattagaagtaacttggtgctatgctagtaagcagtagggaaaaagcagaatcttatcaggataaatggtacaaaatccatatgtcattttccattctccagaatcagccttgtaattctactaaatttacaactctaaataaagtattctgatggcaatacattgttcaattatggctctttctctgactcagaaaaaatcatgattttatcaagttgaatactttttcacctggggggaggtgatttatagtcactagtttgctttaagatgagcctctatgggtactttttacttttattttctgtcattaattgactggcccaggtacgtgtaaagcaggagtgtccaaattttgggttccctgggccacattggaagaagaagaattgtcttgggccacacataaaatacactaacaacagttgatgagctaaaaaaaattgcaaaaagaaattcattatgtttttagaaagtttatgaatttgtgttgggctgcattcaaggccatcttgggccacatgcaacccacagaccgtgggtaggacaagctttgtgtagagtctatgagccagatcttctggctacctcttcagcaaaatctgctgttaaacactggcttgatttggtgaattaggttcagagacagctgcaagctcaacctggtaccctaacgattcgtgtagcatacacataatattcttagtatgatgttaatgagtctatcatgaagaaaacattcctcttatgcaggtacaaatatgagagatattctccttcctccaattccactctcagcatctctccgtgtctcctagagtcaagcagtcaattaccagtttcagttatggaaaaatcaaaatccaaaataagtaacg

(19) United States
(12) Patent Application Publication
(10) Pub. No.: US 2016/0160279 A1
(43) Pub. Date: Jun. 9, 2016

(54) PROGNOSIS OF RESPONSE TO TREATMENT WITH ANTI-TNF-ALPHA IN PATIENTS WITH RHEUMATOID ARTHRITIS

(71) Applicant: Fundacio Hospital Universitari Vall D'Hebron- Institut De Recerca, Barcelona (ES)

(72) Inventors: SARA MARSAL BARRIL, Barcelona (ES); Antoni Julia Cano, Barcelona (ES); Jesus Tornero Molina, Madrid (ES)

(73) Assignee: Fundacio Hospital Universitari Vall D'Hebron- Institut De Recerca, Barcelona (ES)

(21) Appl. No.: 14/930,607

(22) Filed: Nov. 2, 2015

Related U.S. Application Data
(63) Continuation of application No. PCT/ES2014/070377, filed on Apr. 30, 2014.

(30) Foreign Application Priority Data
May 3, 2013 (ES) P201330650

(51) Int. Cl. C12Q 1/68

(52) U.S. Cl. CPC C12Q 1/6883; C12Q 2600/156; C12Q 2600/106

(57) ABSTRACT

The invention relates to the use of SNP rs3794271, and/or an SNP that is in total linkage disequilibrium with same, as a marker in predicting the response to treatment with anti-TNF in a patient with RA. The invention also relates to methods for predicting the response to treatment with anti-TNF, as well as for deciding on or recommending a treatment for a patient with RA, based on determining the genotype for rs3794271 and/or an SNP that is in total linkage disequilibrium with same.

### CROSS REFERENCE TO RELATED APPLICATIONS

This application is a continuation of PCT/ES2014/070377, filed Apr. 30, 2014, which application claims priority of ES P 201330650, filed May 3, 2013, the disclosures of which are hereby incorporated by reference in their entirety for all purposes.

### FIELD OF THE INVENTION

The present invention is related to the rheumatology field, more specifically, with the response to tumor necrosis alpha factor inhibitors in patients with rheumatoid arthritis.

### BACKGROUND OF THE INVENTION

Rheumatoid arthritis (herein referred as RA) is a systemic autoimmune disease, characterized by the presence of a chronic inflammation mainly in the joints. This chronic inflammation is responsible for the progressive destruction of the joints, leading to different degrees of deformity and/or functional impairment. In certain patients, the disease can also have an extraarticular behavior, affecting different organs or systems like the eyes, lungs, pleura, heart and pericardium, skin or blood vessels. Although RA has a yet unknown cause, autoimmunity is known to play a central role in the disease chronification and development. RA can be a very painful and disabling disease.

Approximately 1% of the world's population has RA, being up to three times more frequent in women than in men. Although it can appear at any age, it is more prevalent between the fourth and fifth decades of life.

Tumor necrosis factor alpha inhibitors (herein referred as anti-TNF alpha agents), such as etanercept, adalimumab and infliximab, are proving to be highly successful in the treatment of patients with RA. These drugs are able to slow down the progression of the inflammatory process that perpetuates the disease and, therefore, they are able to reduce the disease activity and significantly improve the quality of life of patients.

Despite this improvement in RA treatment, approximately 20 to 40% of RA patients do not respond significantly to anti-TNF alpha agents or they are unable to maintain the initial positive response to these treatments. The group of patients that do not respond to anti-TNF therapy could therefore benefit from alternative drugs with different mechanisms of action like tocilizumab, abatacept or rituximab. It is now well established that an intense treatment at earlier stages of the disease can be crucial for the positive evolution of patients with RA. For this reason, it would be of high clinical value to be able to predict those patients that will not respond to this treatment. An early prognosis of anti-TNF alpha agent response would help to discard said treatments and allow to use an alternative therapy, which would be highly beneficial for patients since the likelihood of a positive treatment response will be increased and the side effects associated to anti-TNF alpha agents will be avoided. Also, from an economic perspective, and given the high costs of RA treatments, the optimization of clinical decision making and treatment selection would be associated to a significant reduction in the associated health care spending.

Previous studies have analyzed the association of several markers with the response to anti-TNF therapy. Some of these include, the polymorphisms in the promoter region of the gene codifying for the TNF alpha cytokine, as well as polymorphisms in the HLA-DR gene (major histocompatibility complex, class II, DR), FCGR3 (Fc receptor gamma III) and IL1RN (interleukin 1 receptor antagonist) genes. However, the results obtained from these studies have been so far contradictory. More recently, some genome-wide association studies (GWAS) have found certain polymorphisms associated with the response to anti-TNF alpha (Liu C, et al, “Genome-wide association scan identifies candidate polymorphisms associated with differential response to anti-TNF treatment in rheumatoid arthritis”, Mol Med, 2008, vol. 14, pp. 25575-81; Plant D et al, “Genome-wide association study of genetic predictors of anti- tumor necrosis factor treatment efficacy in rheumatoid arthritis identifies associations with polymorphisms at seven loci”, Arthritis Rheum, 2011, vol. 63, pp. 645-53; Krintel SB, et al, “Investigation of single nucleotide polymorphisms and biological pathways associated with response to TNF alpha inhibitors in patients with rheumatoid arthritis”, 30 Pharmacogenet Genomics, vol. 22, pp. 577-89). However, none of these polymorphisms has demonstrated a sufficiently reliable predictive ability. Ultimately, there are yet no markers that have sufficient reliability or robustness to be used in the clinical practice.

For all these reasons, it is necessary to identify markers for prediction of response to anti-TNF agents that can be used in the clinical practice and allow to reliably identify those patients with a negative response to anti-TNF therapies.

### DETAILED DESCRIPTION OF THE INVENTION

The inventors have found that single nucleotide polymorphism (herein referred as SNP) rs3794271 has a high ability to predict the response to anti-TNF alpha therapy in patients with RA. Surprisingly, the research performed by the inventors demonstrate that the association of said SNP with the response to anti-TNF alpha therapy is significant at the genome-wide level. All these experiments are shown in the experimental section of the present document. Based on these results, it can be concluded that SNP rs3794271 is a very powerful marker to predict the response of RA patients to this group of drugs.

Thus, one aspect of the invention refers to the use of SNP rs3794271 as a predictive marker of the response to anti-TNF alpha treatment in a patient with RA.

The examples below show that SNP rs3794271 predicts the response to treatment more accurately when the anti-TNF agent is infliximab or etanercept. Therefore, in a specific embodiment, the invention refers to the use of SNP rs3794271 as a marker in the prediction of the response to treatment with infliximab or etanercept in a patient with RA.

Compared to previous markers described in the state-of-the-art, rs3794271 has shown to have a sufficiently high predictive ability to be used as a tool for decision making in the clinical practice, thereby giving a highly valuable information for the optimization of the pharmacological treatment for each RA patient.

The term “single nucleotide polymorphism” refers to a variation in the DNA sequence that affects a single base (adenine (A), timine (T), cytosine (C) or guanine (G)) of the sequence of the genome. In some instances, however, in the present state-of-the-art, changes in a low number of nucleotides as well as small insertions and deletions (indels), can also be considered as SNPs. If the SNP has a frequency higher than 1% in the general population it is considered a common variant, while it is considered a rare variant if the frequency in the population is lower than 1%. In the present invention, the terms “SNP” and “single nucleotide polymorphism” are used indistinctively, and include both single nucleotide polymorphisms as well as variations that include changes in a few nucleotides, and small deletions or insertions.

SNP rs3794271 is found in the fourth intron of SLCO1C1 gene in human chromosome region 12p12.2 (NCBI reference sequence of chromosome 12: NC_000012.11, publication date: 31 Aug. 2012) and defines genetic variant in position 20,860,093 of said chromosome (according to GRCh37/hg19 human genome assembly, February 2009).

Given that human beings are diploids (that is, they have two sets of autosomal chromosomes in each cell), any polymorphism will be present in both chromosome, maternal and paternal. Each SNP has two single-base sequences which are called “alleles”. In relation to SNP rs3794271, this means that an individual can have the G allele in both chromosomes (i.e. G homozygous individual for this SNP, or GG individual). Alternatively, the individual could have the alternative form A in both chromosome in the SNP location, that is, two A alleles for this SNP (i.e. A homozygous individual, or AA individual), or it could also have one allele of each type (i.e. heterozygous individual, GA or AG).

The inventors have found that, in patients with RA, the presence of the G allele in SNP rs3794271 position indicates a bad response to treatment with either infliximab or etanercept. According to the results of the inventors, heterozygous individuals for this SNP (i.e. one G allele) have a high likelihood of no response to infliximab or etanercept. In the case of GG homozygous patients, this likelihood of no response is even bigger. The genotyping of SNP rs3794271 therefore allows a high accuracy prediction of the response of a patient with RA to the treatment with these drugs.

Thus, another aspect of the present invention refers to an in vitro method for the prediction of the response to an anti-TNF alpha treatment in a patient with RA. This method comprises the determination, from a sample obtained from the patient, of rs3794271 SNP genotype, where the presence of, at least, one G allele (in rs3794271 position) is indicative of a bad response to treatment. This same aspect could be formulated as an in vitro method to predict the response in a RA patient to anti-TNF alpha treatment which comprises determining, from a sample obtained from the patient, the genotype for rs3794271 SNP, where the presence of two alleles other than G in rs3794271 SNP position, is indicative of a good response to treatment. In other words, the presence of the alternative form A in both alleles, that is the presence of two A alleles in the position of SNP rs3794271, indicates a high likelihood of good response to treatment.

In a specific embodiment, the in vitro method of this invention is designed to determine the response to an anti-TNF alpha treatment, selecting between infliximab or etanercept.

In another particular embodiment, the predisposition to a bad response to infliximab or etanercept is associated to the presence of the two G alleles (in rs3794271 SNP position), that is, the predisposition to a bad response is associated to individuals that are GG homozygous in the SNP position.

“Bad response” refers to patients that do not respond to the treatment. “Good response” refers to patients that respond positively to treatment. Treatment response can be determined using known methods in the state-of-the-art, such as the EULAR response (Fransen J et al, “The Disease Activity Score and the EULAR response criteria”, Clin Exp Rheumatol, 2005, vol. 23 (Supl. 39)). The EULAR response classifies patients that are under a specific therapy as good, moderate or non-responders. In clinical studies, patients with a good or moderate response have shown to have a significant improvement in the functional capacity and a reduced progression of joint damage, while non-responder patients do not show a significant reduction in disease activity.

The EULAR response criteria use the DAS28 to determine the response category. The DAS28 is score that is obtained after counting the presence of swelling and/or pain in 28 specific joints, and integrating this value with the erythrosedimentation rate (ESR) and, optionally, the patient's global assessment using a visual scale that ranges from 0 to 100. In a continuous scale that goes from 0 to 9.4, the level of activity of the disease according to DAS28 is considered Low (DAS28 5 3.2), Moderate (3.2<DAS28≦5.1), or High (DAS28>5.1). The EULAR response classifies patients based on the magnitude of the DAS28 change from the basal point to the endpoint, as well as the activity level at the endpoint. A reduction of 1.2 (2 times the measurement error) in the DAS28 of an individual patient is considered a significant change. This way, a patient that has a significant change (DAS28 reduction>1.2) and also reaches a low level of activity (endpoint DAS28≦3.2) is classified as a good responder. If a patient does not reduce the DAS28 in more than 0.6, no matter which is the endpoint DAS28, it is considered a non-responder to therapy. Also, patients that show a DAS28 reduction between 0.6 and 1.2 but their endpoint DAS28 is higher than 5.1, are also considered non-responder patients. The rest of patients, that its, those who do not enter in the EULAR good or EULAR non-responder categories, have an intermediate response and are categorized as moderate responders.

Hence, with regards to the EULAR criteria, the predisposition to a bad response indicates that the patient is an EULAR non-responder, while the predisposition to a good clinical response indicates that the patient is a good or moderate EULAR responder. In a particular embodiment of the present invention, the predisposition to a good responder indicates that the patient is a good EULAR responder.

The method in the present invention provides the medical specialist a very useful tool to decide which is the most adequate pharmacological treatment for a patient with RA. According to the method in this invention, the specialist will be able to discard the treatment with infliximab and etanercept in a specific patient classified as predisposed to a bad response, and recommend an alternative treatment which will have a higher likelihood of response in said patient. The patient will therefore avoid being treated with an ineffective treatment, saving a valuable time for his/her recovery and avoiding side effects associated with the treatment with infliximab or etanercept. Also, according to the method of this invention, those patients that have predisposition to a good response (i.e. those patients that present two different alleles other than G for SNP rs3794271), will benefit from the treatment with infliximab or etanercept.

An aspect of the invention refers to a method to decide or recommend a treatment for a patient with RA which comprises the determination of SNP rs3794271 SNP. If the patient presents, at least, one G allele copy in rs3794271 SNP position, that is if the patient has a bad response predisposition to treatment, a therapy other than anti-TNF alpha agents will be recommended. In a particular embodiment, the anti-TNF agents that will be excluded are infliximab and etanercept. In a particular embodiment, the therapy that excludes infliximab and etanercept is recommended if the patient has the G polymorphic variant in both alleles, that is, if the patient is GG homozygous for SNP rs3794271. In another embodiment, if the patient presents two alleles different than G (generally two A alleles) in rs3794271 SNP position, that is, if the patient has a predisposition to a good response to treatment, a therapy that includes infliximab or etanercept is included.

In a particular embodiment, the method to decide or recommend a treatment for a patient with RA according to the invention comprises, also, the determination of clinical variables of the patient and/or the determination of other biological markers.

It is generally accepted that genetic variations are never isolated in the genome. Given that SNPs are part of a continuous DNA chain (i.e. chromosome), there will be other close variants in the chromosome that will be co-inherited with a very similar frequency and, therefore, will contain a similar level of information to the polymorphism of interest. These close variants that are in high correlation are known as linkage disequilibrium blocks. These blocks, that characterize the whole human genome and have been characterized by the international HapMap project, are clearly defined regions that show a low probability of recombination. SNP rs3794271 is located in a linkage disequilibrium block, defined in SEQ ID NO: 1, and extends from exon 13 of the transcribed sequence of PDE3A gene (NCBI reference sequence: NM_001244683, reference sequence NC_000012.11, GRCh37.p10 primary assembly, 30 Oct. 2012) up to the sixth exon of SLCO1C1 transcribed sequence (NCBI reference sequence: NM_001145945, reference sequence NC_000012.11, GRCh37.p10 primary assembly, 30 Oct. 2012), and including the intergenic region between the two genes. Within this region it is possible to find SNPs that are in very high linkage disequilibrium with rs3794271 SNP, that is, SNPs whose alleles have a very significant correlation with rs3794271 alleles. Linkage disequilibrium is a measure of correlation between the allelic frequencies of two polymorphic genetic loci located in the same chromosome. In most cases, linkage disequilibrium extends up to very short distances, generally in the kilobase range. The region defined by SEQ ID NO:1 constitutes a bad response locus to the treatment with anti-TNF (preferentially, infliximab and etanercept), given that an expert in the area will understand that the SNPs that are in linkage disequilibrium with rs3794271 in this region represent the same response prediction ability than rs3794271.

Therefore, the present invention also contemplates the use of a SNP that is in linkage disequilibrium with rs3794271 as a marker of response prediction to treatment with an anti-TNFalpha agent in a patient with RA. In a particular embodiment, the anti-TNF alpha agent is infliximab or etanercept. Furthermore, the invention contemplates an in vitro method to predict the response of a patient with RA to the treatment with an anti-TNF alpha agent which comprises the determination, from a sample obtained from the patient, of the genotype of a SNP that is in linkage disequilibrium with rs3794271, where the presence of, at least, one allele correlated with rs3794271 G allele is indicative of bad response to treatment. This same aspect could be formulated as an in vitro method to predict the response of a patient with RA to the treatment with an anti-TNF alpha therapy that comprises the determination, form a sample obtained from the patient, of the genotype of a SNP that is in linkage disequilibrium with rs3794271, where the presence of two alleles non-correlated with allele G of rs3794271 is indicative of good response to treatment. In a particular embodiment, the selected anti-TNF alpha is infliximab or etanercept.

By “correlated allele” we understand the allele (polymorphic form) that is in linkage disequilibrium with allele G (polymorphic form G) at rs3794271 (r^{2>}50%). This means that the G allele at SNP rs3794271 and the allele correlated with it, will be coinherited at a very high probability.

Preferentially, the SNP in linkage disequilibrium according to the invention is found in the locus for predisposition a to a bad response to infliximab or etanercept, defined by SEQ ID NO: 1.

In a particular embodiment of the invention the SNP in linkage disequilibrium with SNP rs3794271 is selected from the group of SNPs defined in Table 3. In another particular embodiment, the SNP in linkage disequilibrium is a SP that is in high linkage disequilibrium with rs3794271. By SNP in “high linkage disequilibrium” we refer to those SNPs whose linkage disequilibrium coefficient in relation to rs3794271 is higher than 0.8 (see Table 3).

The invention also contemplates the combination of SNPs that are in linkage disequilibrium with rs3794271 as markers for the prediction of the response to treatment with anti-TNF alpha, preferentially infliximab or etanercept in a patient with RA, as well as an in vitro method to predict the response of a patient with RA to treatment according to the invention that comprises the determination of the genotype in a combination of SNPs that are in linkage disequilibrium with rs3794271. In certain embodiments, rs3794271 is combined with, at least, one SNP in linkage disequilibrium with rs3794271, preferentially a SNP that is located in the locus for predisposition for bad response to infliximab or etanercept defined in SEQ ID NO: 1 and that are reflected in Table 3. In other embodiments the SNPs to combine will be selected from the group consisting of rs10770707, rs1473993, rs10770704, rs10770702, rs959346, rs3751218, rs11045392, rs11045390, rs74406912, rs954866 y rs10770701. In particular embodiments, the combination comprises rs3794271 and rs11045392.

The sample necessary to implement the method of the invention can be any tissue or fluid obtained from the body of the patient, for example, blood, serum, plasma, saliva, urine or hair. In a particular embodiment of the invention the sample is blood or saliva. In another particular embodiment, the sample obtained from the patient is a sample of DNA. However, the method of the present invention does not comprise the sample collection step from the patient.

In a particular embodiment, the patient is a population selected from the group that consists in Caucasian population, Asian population and Centroamerican population.

The state-of-the-art of the technique provides different methods to determine the genotype in a specific region. For example, in a particular embodiment, the determination of the genotype comprises the amplification of the nucleic acid chain region where the SNP is located, for example, through the use of the polymerase chain reaction (PCR). The state-of-the-art comprises methods to design primers to amplify any desired region within the known nucleic acid chain. Primer design can be done automatically using bioinformatic programs that are available for the expert in the area.

In a particular embodiment, the determination of the genotype comprises the sequencing of all or part of the nucleic acid chain of the collected sample. In another embodiment, the genotype determination comprises the hybridisation of the nucleic acid chain of the sample with specific probes. Preferentially, hybridisation will be performed in high specificity conditions.

In another embodiment, the determination of the genotype will be performed using one or more DNA analysis techniques selected form the group consisting of restriction fragment-length polymorphisms (RFLP), random amplification of polymorphic DNA (RAPD), amplified fragment length polymorphisms (AFLPD), all of which are genotyping techniques that are well known for the expert in the area.

The determination of the genotype can require several of the aforementioned techniques. For example, the genotype determination can be performed using amplification and hybridization, or using amplification and sequencing, or using amplification and RAPD. The expert in the area will determine in each case which is the most appropriate technique to determine the genotype.

In another aspect, the present invention refers to the use of methods to determine the genotype for rs3794271, and/or at least one SNP that is linkage disequilibrium with rs3794271 in an in vitro method as has been previously described, in order to predict the response of a patient with RA to the treatment with an anti-TNF alpha agent, preferentially, an anti-TNF alpha agent selected between infliximab or etanercept. In a particular embodiment, the means are hybridization probes specific for rs3794271 or for a SNP that is in linkage disequilibrium with rs3794271. In another particular embodiment, the means are primers specific for SEQ ID NO:1 amplification or any region comprised within SEQ ID NO:1. In another particular embodiment, the means are specific primers in order to perform the determination of the genotype using RAPD.

An additional aspect of the invention refers to a kit to predict the response to treatment with an anti-TNF alpha agent in a patient with RA that comprises means to determine the genotype for rs3794271 SNP, and/or at least one SNP that is in linkage disequilibrium with rs3794271, as well as instructions to carry out said determination and for the interpretation of the results, where these instructions for the interpretation of the results indicate that the presence of at least one G allele for SNP rs3794271 and/or the presence of, at least, a correlated allele with allele G of rs3794271 for the SNP in linkage disequilibrium is indicative of bad response to treatment. Alternatively, or together with the previous, the instructions for the interpretation of the results could indicate that the presence of two alleles other than G for SNP rs3794271 and/or the presence of the two alleles non-correlated with allele G of rs3794271 in the SNP in linkage disequilibrium is indicative of good response to treatment. In a particular embodiment of this aspect of the invention, the kit predicts the response to an anti-TNF alpha selected between infliximab or etanercept.

In a particular embodiment, the kit comprises the means for determining the genotype of rs3794271 SNP as well as instructions to perform said determination and for the interpretation of the results, where these instructions for the interpretation of the results indicate that the presence of two G alleles at SNP rs3794271 is indicative of bad response to treatment.

Throughout the description and claims, the word “comprise” and its variants do not pretend to exclude other technical characteristics, additives, components or steps. Also, the word “comprise” includes the case “consists of”. For those experts in the area, other objects, advantages and characteristics of the invention will be derived in part from the description and in part from the practice of the invention. The following examples will be provided as an illustration, and are not intended to limit the present invention. Also, the present invention covers all the possible combinations of particular and preferred embodiments herein indicated.

### EXAMPLES

Patients

A total of 315 RA patients were included in the present study. All patients were collected as part of the Immune-Mediated Inflammatory Disease Consortium (IMIDC). The IMIDC is a network of Spanish researchers working on the genomic basis of immune-mediated inflammatory diseases. RA patients were collected from the outpatients' clinics of the rheumatology departments from 12 Spanish University Hospitals. The selection criteria were as follows: a) fulfillment of the 1987 American College of Rheumatology classification criteria for RA b) >18 years old c) Caucasian European born in Spain d) all four grandparents born in Spain e) >2 years of follow-up since diagnosis f) having received an anti-TNF therapy (infliximab, etanercept or adalimumab) as the first biologic treatment and g) baseline DAS28 score ≧3.2.

Informed consent was obtained from all participants and protocols were reviewed and approved by local institutional review boards. The present study was conducted according to the Declaration of Helsinki principles.

### Treatment Response Definition

The response to anti-TNF alpha treatment was measured using the absolute and relative DAS28 changes, as well as the EULAR response. The endpoint DAS28 was measured at 12 weeks of treatment. The absolute DAS28 change (ΔDAS28) is defined as the change in DAS28 between baseline and endpoint scores (DAS25_{baseline}-DAS28_{week12}); EULAR divides the response to anti-TNF alpha into three categories: good, moderate and non-responder.

DNA Extraction, SNP Selection and Genotyping

Genomic DNA was isolated from venous blood samples using the Chemagic

Magnetic Separation Module I (PerkinElmer). A total of 4 SNPs were selected for genotyping: i) 2 SNPs associated with ΔDAS28: rs10520789 at ˜727 kb from the 5′ end of nuclear receptor subfamily 2, group F, member 2 (NR2FR2) gene (chromosome 15q26.2), rs11870477 at ˜264 kb from the 3′ end of mitogen-activated protein kinase kinase 6 (MAP2K6) gene (chromosome 17q24.3), ii) 1 SNP associated with reIDAS28: rs1539909 at ˜955 kb from the 3′ end of cerebellin 2 precursor (CBLN2) gene (chromosome 18q22.3), iii) 1 SNP associated with EULAR response: rs3794271 in the fourth intron of solute carrier organic anion transporter family, member 1C1 (SLCO1C1) gene (chromosome 12p12.2).

SNP genotyping was performed using the TaqMan genotyping platform (Life Technologies). The selected Taqman assays were: C_30397748_20 (rs10520789, NR2FR2), C_32133788_10 (rs11870477, MAP2K6), C_7536165_10 (rs1539909, CBLN2) and C_27502188_10 (rs3794271, SLCO1C1). Thermal cycle conditions were as follows: 50° C. for two minutes and 95° C. for 10 minutes, followed by 40 cycles of 92° C. for 15 seconds and 60° C. for one minute. All PCR and end point fluorescent readings were performed using an ABI PRISM7900 HT sequence detection system (Life Technologies). The genotyping error was estimated by genotyping 10% of the samples in duplicate.

### Statistical Analysis

Multivariate linear regression was used to test the association with the absolute change in Disease Activity Score (ΔDAS28) using the baseline DAS28 as a covariate. The allelic X² test was used to test for association with the European League Against Rheumatism (EULAR) extreme response. Bonferroni adjustment of the significance level was performed to account for multiple testing (α=0.0124). All association analyses were performed using R statistical software (http://cran.r-project.org/) Power calculations were performed using Quanto (v 1.2.4, http://hydra.usc.edu/gxe/) and the online Genetic Power Calculator (http://pngu.mgh.harvard.edu/˜purcell/gpc/). All SNPs were tested for deviations from Hardy-Weinberg Equilibrium.

In order to investigate the effect of potential confounders of the observed genetic association with EULAR response, a logistic regression model was fitted. Evaluated variables were age at diagnosis, age at anti-TNF treatment, disease duration, baseline DAS28, smoking habit, gender, number of erosions, presence of Rheumatoid Factor (RF) and presence of anti-cyclic citrullinated peptide antibodies (anti-CCP). The presence of a drug type-specific effect was also evaluated by fitting a logistic regression model including an interaction term between SNP genotype and anti-TNF therapy type as described in Plant et al (supra).

### Results

Using a cohort of 315 RA patients four candidate loci previously identified in a GWAS for anti-TNF response were tested. The call rate for the four SNPs was >99% and the genotyping error was 0%. All four SNPs were in Hardy-Weinberg Equilibrium (P>0.5). The clinical features of our patient cohort are shown in Table 1.

TABLE 1
Clinical characteristics of patients

| | |
|---|---|
| Age at diagnosis, mean ± SD | 43 ± 12 |
| No. (%) female | 257(81) |
| Disease duration (years), mean ± SD | 11 ± 8 |
| No. (%) Anti-CCP positive* | 239(79) |
| No. (%) RF positive* | 246(78) |
| Baseline DAS28, mean ± SD | 5.5 ± 1.1 |
| No. (%) treated with infliximab* | 115(36) |
| No. (%) treated with etanercept* | 113(36) |
| No. (%) treated with adalimumab | 87(28) |
| Smoking habit^{a} | 78(25) |
| No. (%) EULAR None Responders^{b} | 77(24) |
| No. (%) EULAR Good Responders^{b} | 105(33) |

^{a}Current smokers.
^{b}Determined at week 14

In the EULAR good vs. none response a highly statistically significant association between PDE3A-SLCO1C1 SNP rs3794271 with treatment response was found (P=1.74E-5, OR (95% CI)=2.63 (1.68-4.12). Furthermore, adding the EULAR moderate group of anti-TNF responders to the good responder group, the association was still highly significant (P=2.69E-4, OR (95% CI)=1.98 (1.37-2.89)). None of the other tested markers showed significant association even at the nominal level (P<0.05). rs3794271 genotype frequencies for all three EULAR categories and average ΔDAS28 and reIDAS28 scores are shown in Table 2.

TABLE 2
Genotypic frequencies of rs3794271 for the three
EULAR categories and the average ΔDAS values.

| | s3794271 genotype | | | | | |
|---|---|---|---|---|---|---|
| | AA | | AG | | GG | |
| ULAR | umber | DAS | umber | DAS | umber | DAS |
| response | (%) | (mean ± SD) | (%) | (mean ± SD) | (%) | (mean ± SD) |
| ULAR GOOD | 8 (18.5) | .7 ± 1.0 | 1 (13.1) | .7 ± 1.0 | (1.6) | .4 ± 0.6 |
| ULAR MODE RATE | 3 (16.9) | .7 ± 0.8 | 7 (21.4) | .6 ± 0.7 | 2 (6.4) | .9 ± 1.0 |
| ULAR NONE | 0 (6.4) | .0 ± 1.1 | 3 (13.7) | .1 ± 0.7 | 4 (4.5) | 0.2 ± 1.0 |

The results demonstrate that SNP rs3794271 is associated with the prediction of the response to anti-TNF alpha therapy. The ability of the SNP to predict those patients that are bad responders according to EULAR criteria (compared to good responders) is very high, and also to predict those patients that are bad responders according to the EULAR criteria (differentiating them from good and moderate).

In a secondary analysis, it was evaluated the observed genetic association after controlling for potential confounders. For any clinical variable analyzed as a potential confounder, the change in the SNP regression coefficient and statistical significance was negligible. This result confirms that the relation between the SP and the anti-TNF response is stable and independent of any clinical variable. Also, the presence of an interaction with each of the clinical covariates was tested by adding an additional interaction term to the logistic regression model, but none was found to be significant. This result confirms that the SNP association does not interact with any other clinical variable. However, testing for drug type-specific effects a significant interaction between the presence of adalimumab and anti-TNF response was found (P =0.045). Using the Breslow-Day method to test for differences in the association between adalimumab treated patients against the other two treatments a suggestive evidence of heterogeneity was found (P =0.078). Testing each treatment separately, no significant association of SNP rs3794271 with response in adalimumab-treated patients was found (P =0.37), while there was substantial evidence of association for infliximab (P=0.0046) and etanercept (P=0.00028). These results indicate that the ability of SNP rs3794271 to predict the response to anti-TNF therapy is more accurate when it refers to anti-TNF drugs infliximab and etanercept.

Additionally, a meta-analysis was performed to combine the association evidence between SNP rs3794271 with the response to anti-TN F alpha therapy from the present study with that from the previous GWAS performed by Krintel et al. (supra). The fixed-effects model implemented in the “rmeta” R package was used to estimate the summary Odds Ratio (OR). The METAL software tool (http://www.sph.umich.edu/csg/abecasis/metal/index.html) to calculate the combined P-value. In this approach, the significance values are weighted according to the sample size of each study. ORs were calculated according to the minor allele and to the risk of being EULAR none responder. Variance in liability explained by PDE3A-SLCO1C1 SNP was estimated in the replication sample using the approach proposed by So et al (“Evaluating the heritability explained by known susceptibility variants: a survey of ten complex diseases”, Genet Epidemiol, 2011, vol. 35, pp. 310-7). Combining the statistical evidence from the GWAS and the present association study, a genome-wide significant association of PDE3A-SLO1C1 SNP rs3794271 with anti-TNF treatment response was found (P =3.34E-10). The estimated summary effect size for this genetic association was OR(95% CI) =2.91 (2.57-3.25).

We evaluated the allele dose effect by comparing the effect size with one or two copies of the minor allele (G). The effect size for the heterozygote genotype was OR(95% CI) =3.0 (1.6-5.9). The effect size for the homozygote was even bigger OR(95% CI) =8.1 (3.3-19.7). These results indicate that the association between a bad response to anti-TNF alpha treatment for heterozygote individuals at the rs3794271 SNP position is very high, and that this association is even bigger for GG homozygous individuals.

A study of the genomic region that harbors rs3794271 was also performed.

This study revealed the existence of two peaks of high recombination levels (“hotspot” regions) in the 13th exon of the transcribed sequence of PDE3A gene and in the sixth exon of the transcribed sequence of gene SLCO1C1, which delimit a linkage disequilibrium block that is clearly differentiated from the rest of the genome. This block, defined by SEQ ID NO: 1, concentrates SNPs that are in high linkage disequilibrium with rs3794271. These results indicate that the linkage disequilibrium block defined by SEQ ID NO: 1 constitutes a locus that predisposes to the response to anti-TNF alpha treatment. The sequence corresponding to the PDE3A gene that is included in this predisposing locus goes from position 44,910 from sequence number 1 to position 61,065. SEQ ID NO: 1 contains also the intergenic sequence between PDE3A and SLCO1C1 (from position 33,663 to position 44,909). SNP rs3794271 is located in position 56,714 from SEQ ID NO: 1.

Table 3 includes those SNPs from the sequence that predisposes to a bad response to treatment (SEQ ID NO: 1) and that are in linkage disequilibrium with rs3794271, as well as the minor allele (A1), the major allele (A2) and the allele correlated with variant G (risk allele) for each of them. The risk allele is the polymorphic form in the location of each of the SNPs that is in linkage disequilibrium with the G allele for of SNP rs3794271. Risk alleles, therefore, yield the same information with regards to the predisposition to treatment response.

TABLE 3
SNPs in linkage disequilibrium with SNP rs3794271 in the locus for the
predisposition to bad response to infliximab or etanercept treatment.

| Position in | | | | | | |
|---|---|---|---|---|---|---|
| SEQ ID1 | | Position in | | | | Risk |
| NO: 1 | Reference | chromosome 12 | LD¹ | A¹ | A² | Allele^{2,} ³ |
| 7739 | rs4451779 | 20811118 | 0.60 | T | G | T |
| 13643 | rs10743388 | 20817022 | 0.66 | C | G | C |
| 14099 | rs10770689 | 20817478 | 0.60 | T | C | T |
| 15772 | rs6487129 | 20819151 | 0.58 | G | A | G |
| 17061 | rs10770691 | 20820440 | 0.58 | C | G | C |
| 17492 | rs11045376 | 20820871 | 0.56 | C | T | C |
| 18105 | rs11045378 | 20821484 | 0.57 | T | A | T |
| 24396 | rs10770695 | 20827775 | 0.63 | T | C | T |
| 25316 | rs12301364 | 20828695 | 0.61 | A | G | A |
| 26141 | rs10841593 | 20829520 | 0.61 | G | A | G |
| 27013 | rs151131008 | 20830392 | 0.61 | TTTTTC | T | TTTTTC |
| 28335 | rs11045385 | 20831714 | 0.62 | T | C | T |
| 29216 | rs10770701 | 20832595 | 0.81 | A | G | A |
| 30640 | rs954866 | 20834019 | 0.85 | A | G | A |
| 34206 | rs3809209 | 20837585 | 0.74 | C | T | C |
| 34785 | rs7305718 | 20838164 | 0.77 | C | T | C |
| 37058 | rs74406912 | 20840437 | 0.85 | GT | G | GT |
| 37436 | rs11045390 | 20840815 | 0.85 | T | C | T |
| 37460 | rs11045392 | 20840839 | 0.84 | T | C | T |
| 37742 | rs2203493 | 20841121 | 0.75 | A | T | A |
| 38709 | rs137927950 | 20842088 | 0.66 | T | TTC | T |
| 40201 | rs2417861 | 20843580 | 0.77 | C | T | 0 |
| 45688 | rs3751218 | 20849067 | 0.95 | G | A | G |
| 48707 | rs959346 | 20852086 | 0.83 | G | A | G |
| 48942 | rs10770702 | 20852321 | 0.83 | T | G | T |
| 50162 | rs10743389 | 20853541 | 0.51 | T | C | T |
| 52382 | rs10770704 | 20855761 | 0.83 | T | C | T |
| 52973 | rs71939085 | 20856352 | 0.59 | TTGA | T | TTGA |
| 53200 | rs10743390 | 20856579 | 0.58 | T | C | T |
| 53894 | rs11392906 | 20857273 | 0.69 | T | TC | T |
| 53895 | rs201855778 | 20857274 | 0.69 | G | GC | G |
| 54088 | rs10770705 | 20857467 | 0.53 | A | C | A |
| 54097 | rs10770706 | 20857476 | 0.69 | G | A | G |
| 54283 | rs78690605 | 20857662 | 0.69 | C | CAGAT | C |
| 54906 | rs10505868 | 20858285 | 0.69 | C | T | C |
| 56271 | rs1473993 | 20859650 | 0.87 | C | T | C |
| 56605 | rs3838816 | 20859984 | 0.54 | AT | A | AT |
| 56714 | rs3794271 | 20860093 | 1.00 | G | A | G |
| 58072 | rs10770707 | 20861451 | 0.99 | A | T | A |

i. ¹LD = linkage disequilibrium coefficient
ii. ²More than 1 nucleotide means that the SNP is an insertion/deletion
iii. ³ Calculated in Caucasian population

In summary, strong evidence was found for the association between the locus defined by genes PDE3A and SLOC1C1, and their intergenic region, particularly the region defined by SEQ ID NO: 1 with the response to anti-TNF alpha treatment in patients with RA. Based on the obtained results, this association is particularly significant for infliximab and etanercept drugs and for GG homozygous patients at position rs3794271.

SLCO1C1 is a cell membrane protein that belongs to the OATP1 organic anion transporting polypeptide family. This group of proteins have a wide substrate specificity and been shown to play an important role in the absorption, distribution and excretion of drugs, including methotrexate. To date, the protein encoded by SLCO1C1 has been mainly associated to thyroid hormone and estradiol transport. However, the fact that this gene encodes for four different transcript variants increases the probability that more functionality will be identified.

PDE3A codes for a phosphodiesterase (PDE) that hydrolyzes important secondary messengers like cAMP and cGMP. Cyclic nucleotide PDEs have shown important immunomodulatory functions and they have been considered themselves as important targets for the management of autoimmune diseases including RA. For example, PDE inhibition has shown to reduce TNF production in Lipoplysaccharide stimulated monocytes.

1. An in vitro method for the prediction of the response of a patient with rheumatoid arthritis (RA) to treatment with tumor necrosis factor inhibitor alpha (anti-TNF alpha agent) selected between infliximab or etanercept that comprises determining, from a sample obtained from the patient, the genotype for single nucleotide polymorphism (SNP) rs3794271, and/or at least one SNP that is in linkage disequilibrium with SNP rs3794271, wherein:
i. the presence of at least one G allele at SNP rs3794271 is indicative of a bad response to treatment while the presence of two alleles other than G at SNP rs3794271 is indicative of a good response to therapy; and/or
ii. the presence of at least one allele correlated with the G allele at SNP rs3794271 for the SNP in linkage disequilibrium is indicative of a bad response to treatment while the presence of two alleles not correlated with the G allele at SNP rs3794271 for the SNP in linkage disequilibrium is indicative of a good response to treatment.

2. The method according to claim 1, where the presence of two G alleles at SNP rs3794271 indicates a predisposition to a bad response to treatment.

3. The method according to claim 1, wherein the genotype is determined for SNP rs3794271.

4. The method according to claim 1, wherein the obtained sample is selected from the group that comprises plasma, blood, serum and saliva.

5. The method according to claim 1, wherein the genotype determination is performed using a DNA analysis technique selected from the group including sequencing, hybridization, Restriction-Fragment Length Polymorphisms, Random Amplification of Polymorphic DNA (RAPD), Polymerase Chain Reaction (PCR) or Amplified Fragment Length Polymorphisms (AFLPD) and a combination thereof.

6. A method to decide or recommend a treatment for a patient with RA that comprises the determination of a SNP rs3794271 genotype and/or a SNP that is in linkage disequilibrium with rs3794271 according to claim 1, wherein if the patient has a predisposition to a bad response to treatment, a therapy that excludes infliximab and etanercept is recommended.

7. A method to decide or recommend a treatment for a patient with RA that comprises the determination of a rs3794271 SNP genotype and/or a SNP that is in linkage disequilibrium with rs3794271 according to claim 1, wherein if the patient has a predisposition to a good response to treatment, a therapy that includes infliximab or etanercept is recommended.

8. The method according to claim 6, wherein the genotype for SNP rs3794271 is determined, and if the patient presents at least one G allele, a therapy that excludes infliximab or etanercept is recommended.

9. The method according to claim 7, wherein the genotype for SNP rs3794271 is determined, and if the patient presents two alleles other than G, a therapy that includes infliximab or etanercept is recommended.

10. The method according to claim 1, wherein the SNP in linkage disequilibrium is located in a locus for predisposition to a bad response to treatment as defined by SEQ ID NO: 1.

11. The method according to claim 10, wherein the SNP in linkage disequilibrium is selected from the group consisting of rs10841585, rs10841586, rs4451779, rs10743388, rs10770689, rs6487129, rs10770691, rs11045376, rs11045378, rs10770695, rs12301364, rs10841593, rs151131008, rs11045385, rs10770701, rs954866, rs3809209, rs7305718, rs74406912, rs11045390, rs11045392, rs2203493, rs137927950, rs2417861, rs3751218, rs959346, rs10770702, rs10743389, rs10770704, rs71939085, rs10743390, rs11392906, rs201855778, rs10770705, rs10770706, rs78690605, rs10505868, rs1473993, rs3838816 and rs10770707.

12. The method according to claim 11, wherein the SNP in linkage disequilibrium is selected from the group consisting of rs10770707, rs1473993, rs10770704, rs10770702, rs959346, rs3751218, rs11045392, rs11045390, rs74406912, rs10770701 and rs954866.

13. Use of SNP rs3794271, and/or a SNP that is in linkage disequilibrium with SNP rs3794271, as a marker of predisposition to the response to treatment with an anti-TNF alpha agent selected between infliximab and etanercept in a patient with RA.

14. Use according to claim 13, wherein the SNP in linkage disequilibrium is located in a locus predisposing to a bad response to treatment as defined by SEQ ID NO: 1.

15. Use according to claim 14, wherein the SNP in linkage disequilibrium is selected from the group consisting of rs10841585, rs10841586, rs4451779, rs10743388, rs10770689, rs6487129, rs10770691, rs11045376, rs11045378, rs10770695, rs12301364, rs10841593, rs151131008, rs11045385, rs10770701, rs954866, rs3809209, rs7305718, rs74406912, rs11045390, rs11045392, rs2203493, rs137927950, rs2417861, rs3751218, rs959346, rs10770702, rs10743389, rs10770704, rs71939085, rs10743390, rs11392906, rs201855778, rs10770705, rs10770706, rs78690605, rs10505868, rs1473993, rs3838816 and rs10770707.

16. Use according to claim 15, wherein the SNP in linkage disequilibrium is selected from the group consisting of rs10770707, rs1473993, rs10770704, rs10770702, rs959346, rs3751218, rs11045392, rs11045390, rs74406912, rs10770701 and rs954866.

17. Use according to claim 13, wherein the marker is SNP rs3794271.

18. A kit to predict the response to treatment with an anti-TNF alpha agent selected between infliximab or etanercept in a patient with rheumatoid arthritis which includes means to determine the genotype of SNP rs3794271 and/or at least one SNP that is in linkage disequilibrium with SNP rs3794271, and instructions to perform said determination and for the interpretation of the results, wherein said instructions for the interpretation of the results indicate that the presence of at least one G allele at SNP rs3794271 and/or the presence of an allele correlated with the G allele at SNP rs3794271 for the SNP in linkage disequilibrium is indicative of a bad response to treatment, while the presence of two alleles other than G at SNP rs3794271 and/or the presence of two alleles not correlated with the G allele at SNP rs3794271 for the SNP in linkage disequilibrium is indicative of a good response to treatment.

19. The kit according to claim 18, wherein the SNP in linkage disequilibrium is located in a locus predisposing to a bad response to treatment as defined by SEQ ID NO: 1.

20. The kit according to claim 19, wherein the SNP in linkage disequilibrium is selected from the group consisting of rs10841585, rs10841586, rs4451779, rs10743388, rs10770689, rs6487129, rs10770691, rs11045376, rs11045378, rs10770695, rs12301364, rs10841593, rs151131008, rs11045385, rs10770701, rs954866, rs3809209, rs7305718, rs74406912, rs11045390, rs11045392, rs2203493, rs137927950, rs2417861, rs3751218, rs959346, rs10770702, rs10743389, rs10770704, rs71939085, rs10743390, rs11392906, rs201855778, rs10770705, rs10770706, rs78690605, rs10505868, rs1473993, rs3838816 and rs10770707.

21. The kit according to claim 20, wherein the SNP in linkage disequilibrium is selected from the group consisting of rs10770707, rs1473993, rs10770704, rs10770702, rs959346, rs3751218, rs11045392, rs11045390, rs74406912, rs10770701 and rs954866.

22. The kit according to claim 18, wherein the means are used to determine the genotype for SNP rs3794271 and the instructions indicate that the presence of at least one G allele at SNP rs3794271 is indicative of a bad response to treatment, while the presence of two alleles other than G at SNP rs3794271 is indicative of a good response to treatment.
